(19) United States
(12) Patent Application Publication
(10) Pub. No.: US 2013/0331394 A1
(43) Pub. Date: Dec. 12, 2013

(54) TREATING SCHIZOPHRENIA

(71) Applicant: The McLean Hospital Corporation, Belmont, MA (US)

(72) Inventor: Peter Siekmeier, Cambridge, MA (US)

(21) Appl. No.: 13/915,413

(22) Filed: Jun. 11, 2013

Related U.S. Application Data
(60) Provisional application No. 61/658,085, filed on Jun. 11, 2012.

(51) Int. Cl. G01N 33/50; A61K 45/06; A61K 31/198; A61K 31/454; A61K 31/4015; A61K 31/5025; A61K 31/453

(52) U.S. Cl. CPC G01N 33/5088; A61K 31/5025; A61K 45/06; A61K 31/453; A61K 31/454; A61K 31/4015; A61K 31/198
USPC 514/249; 435/29; 514/248; 514/321; 514/322; 514/423; 514/424; 514/561; 800/3

(57) ABSTRACT

Methods of identifying new treatments for schizophrenia, and the use of the same.

### CLAIM OF PRIORITY

This application claims the benefit of U.S. Provisional Application Ser. No. 61/658,085, filed on Jun. 11, 2012, which is incorporated herein by reference in its entirety.

### FEDERALLY SPONSORED RESEARCH OR DEVELOPMENT

This invention was made with Government support under Grant No. K08 MH072771 awarded by the National Institutes of Mental Health of the National Institutes of Health. The Government has certain rights in the invention.

### TECHNICAL FIELD

This application relates to methods of identifying new treatments for schizophrenia, and the use of the same.

### BACKGROUND

Schizophrenia is a debilitating, lifelong illness affecting approximately 1% of the population worldwide [1]. Beginning with Thorazine (chlorpromazine) in the 1950s, antipsychotic medications have been used to treat the condition. However, despite several years of research, and the introduction of a number of new agents, all currently used antipsychotics are far from ideal. They are capable of ameliorating some symptoms in many, though not all, schizophrenic patients, and none represents a cure to the disease. Moreover, these medications carry significant side effect burdens [2].

The absence of development of antipsychotic medications with fundamentally new mechanisms of action [3] stands in stark contrast to the vast amounts that have been learned over the past 20 years on the cellular level abnormalities associated with the disease. The hippocampal neurobiology of the illness has been the subject of a number of recent comprehensive and detailed reviews [4]. Broadly, studies on this and other brain areas have revealed: (1) Dysfunction in the gamma-aminobutyric acid (GABA) system. Deficiencies in GABAergic innervation have been seen, as a result of decreased number of particular subtypes of GABA neurons or GABAergic tone, and a (presumably compensatory) increase in postsynaptic GABA receptor expression [5]; (2) Glutamatergic system deficiency. This is manifested, for example, as decreased expression of N-methyl-D-aspartic acid (NMDA) receptors, and/or hypofunction of NMDA synaptic activity [6,7]; and (3) Decreases in brain connectivity. Diminished dendritic spine density has been seen, for example, in postmortem and animal models of the illness [8,9].

One reason this large and growing body of neurobiological knowledge has not translated into more effective treatments is that a lack convincing causative links between cellular level abnormalities and particular symptoms or sets of symptoms. This is a problem that is characteristic of psychiatric illnesses in general, and stands in contrast to many other medical illnesses, in which, for example, the underlying genetic abnormality, the dysfunctional protein expressed, the function of this protein, and the manner in which this causes illness pathology are well understood. This is made particularly difficult because function in a given region, such as hippocampus, is likely an emergent phenomenon; it is extremely difficult to intuit the behavior of the overall system by looking at one, or even a few of its constituent cellular level behaviors or interactions in isolation [10]. It is difficult to imagine designing an effective intervention without taking this into account.

### SUMMARY

Many traditional drug discovery efforts have involved identifying an abnormality in a cellular level entity (e.g., a receptor, enzyme, or ion channel) associated with an illness, and creating an agent to counteract that particular deficiency. The method described here represents an improvement in two ways. First, it allows for the evaluation of multiple medication effects simultaneously. Second, neuropsychiatric illness is likely the result of system level failure. It may be possible to re-equilibrate the system by affecting in ways that are not simple reversals of the causative lesions. This invention can identify such mechanisms.

Thus, in a first aspect the invention provides methods for identifying a candidate agent for the treatment of schizophrenia. The methods include providing a sample comprising a cell expressing functional 2-amino-3-(3-hydroxy-5-methyl-isoxazol-4-yl)propanoic acid (AMPA) channels; contacting the sample with a test compound; measuring the decay time constant (tau2) of the AMPA conductance (i.e., the current through the AMPA receptor channels) in response to stimulation in the presence, and optionally in the absence, of the test compound; and

selecting as a candidate agent a test compound that decreases the tau2 of the AMPA conductance, i.e., decreases the tau2 as compared to the tau2 in the absence of the test compound, or as compared to a reference or control tau2 that represents tau2 in the absence of the test compound.

In some embodiments, the methods include selecting as a candidate agent a test compound that decreases the tau2 of the AMPA conductance to 3 msec or less.

In some embodiments, the decay time constant is measured electrophysiologically or by imaging of a calcium imaging agent.

In another aspect, the invention provides methods for identifying a candidate agent for the treatment of schizophrenia. The methods include providing a sample comprising a neural network comprising a calretinin-positive (CR+) GABAergic interneuron, and at least one postsynaptic cell, i.e., a neuron or interneuron receiving synaptic input from the CR+ interneuron; contacting the sample with a test compound; stimulating the CR+ interneuron and measuring the response in the postsynaptic cell in the presence and absence of the test compound; and selecting as a candidate agent a test compound that increases the response in the postsynaptic cell (i.e., wherein the response is increased in the presence of the test compound as compared to the response in the absence of the test compound).

In some embodiments, the neural network is a neocortical, allocortical, or hippocampal brain slice, or an in vitro neural network.

In some embodiments, the brain slice is from an animal model of schizophrenia, or from a normal non-schizophrenic animal.

In some embodiments, the in vitro neural network comprises primary neurons from an animal model of schizophrenia, or from a normal non-schizophrenic animal.

In some embodiments, measuring the response in the postsynaptic neuron or interneuron comprises measuring one or more of: long term potentiation at the postsynaptic synapse; short term potentiation; conductance change; response to paired pulses; inhibitory postsynaptic current (IPSC); and inhibitory postsynaptic potential (IPSP) in the postsynaptic cell (neuron or interneuron).

In another aspect, the invention provides methods for identifying a candidate agent for the treatment of schizophrenia. The methods include providing a sample comprising a postsynaptic cell, i.e., a neuron or interneuron that receives synaptic input from a calretinin-positive (CR+) GABAergic interneuron; identifying a combination of GABAA receptor subunits expressed in the postsynaptic cell; selecting a drug that is a specific agonist of GABAA receptors comprising the subunits expressed in the postsynaptic cell as a candidate agent for the treatment of schizophrenia.

In some embodiments, selecting a drug that is a specific agonist of GABAA receptors comprising the subunits expressed in the postsynaptic neuron as a candidate agent for the treatment of schizophrenia includes: expressing the subunits expressed in the postsynaptic neuron or interneuron in a mammalian cell to form functional GABAA receptors; contacting the mammalian cell with a test compound; detecting conductance through a GABAA receptor in the cell in the presence of the test compound; and selecting as a candidate compound a test compound that increases conductance as compared to conductance in the absence of the test compound.

In some embodiments, the methods include administering the selected candidate compound to an animal model of schizophrenia; monitoring one or more symptoms of schizophrenia in the animal model; and selecting as a candidate therapeutic agent a candidate compound that improves one or more symptoms of schizophrenia in the animal model.

In some embodiments, the methods include administering the selected candidate compound to an animal model of schizophrenia; monitoring one or more symptoms of schizophrenia in the animal model; and selecting as a candidate therapeutic agent a candidate compound that improves one or more symptoms of schizophrenia in the animal model.

In some embodiments, the methods include administering the selected candidate compound to an animal model of schizophrenia; monitoring one or more symptoms of schizophrenia in the animal model; and selecting as a candidate therapeutic agent a candidate compound that improves one or more symptoms of schizophrenia in the animal model.

In another aspect, the invention provides methods for treating schizophrenia in a subject, the method comprising administering a therapeutically effective amount of a combination of compounds including (a) an NMDA agonist and a GABAA-alpha 2 agonist; or (b) an NMDA agonist and an AMPAkine.

In some embodiments, the NMDA agonist is selected from the group consisting of UBP646, UBP512, UBP551, CIQ, Glycine, D-cycloserine, glycine type I (GlyT1) transporter inhibitors, and D-serine. In some embodiments, the glycine type I (GlyT1) transporter inhibitor is sarcosine (N-methylglycine) or RG1678.

In some embodiments, the GABAA-alpha 2 agonist is MK-0777, TPA023B or MRK-409.

In some embodiments, the AMPAkine is piracetam, aniracetam, CX516, CX717, CX691 (faramptor), LY451395 or CX546.

The abbreviation AMPA stands for 2-amino-3-(3-hydroxy-5-methyl-isoxazol-4-yl)propanoic acid, a specific agonist for the AMPA receptor.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A-D. Brain oscillatory activity from clinical magnetoencephalographic (MEG) and EEG studies, and output of computational model. (A) Control subjects (left three histograms) and schizophrenic patients (right three histograms) were exposed to auditory click trains at 20, 30, and 40 Hz. Resultant MEG power spectra are shown (from Vierling-Claassen et al [54]). (B) The same experimental conditions as (A) above were used, but EEG activity was recorded (from Kwon et al [41]). (C) Simulated EEG power spectra from model when driven at 20, 30, and 40 Hz. Note correspondence with clinical data of panels (A) and (B). (This is the “primary point model”, as defined in FIG. 2.) (D) Graph of power spectrum peaks from index schizophrenic patient of panel (C) plus 20 simulated patients (in red), and index control patient of panel (C) plus 20 simulated control subjects (blue). In all cases, index patient is indicated by a star; simulated patient averages are indicated by dot, and one and two standard deviations are shown by tick marks on error bar. Although computational model outcomes are not strictly analogous to data from clinical studies [59]-[61], we have calculated p-values, by convention (*p<0.01, **p<0.001). Note that Group×Frequency interaction was highly significant, due to the fact that group differences were largest at 40 Hz; please see text for additional details of statistical analysis.

FIG. 2. Model output showing unique combinations of abnormalities that may give rise to the schizophrenic phenotype.

Degree of GABA system dysregulation, extent of NMDA hypofunction, and spine density decrease are shown on the axes, respectively. Origin (0, 0, 0) represents the control (unaffected) condition. The degree to which model outputs match experimental findings (illness metric) is indicated via color scale. All model outcomes with illness metric >0.65 are shown.

FIG. 3A-C. Response of system with respect to change in single parameters.

Oscillatory activity (power) at 20, 30, and 40 Hz with respect to (A) decreased NMDA activity, (B) decreased pyramidal cell spine density, and (C) increasing GABA defect is shown. Colored tick marks on right border of graphs indicate oscillatory behavior characteristic of schizophrenic patients. Solid lines represent model response at that frequency to drive at the given frequency (e.g., solid blue line represents power of 20 Hz activity when model is driven at 20 Hz). Dashed red line represents 20 Hz response to 40 Hz drive. Resp=response.

FIG. 4. Relative contribution of each component of GABA system deficit.

To understand the relative contribution of each component of the GABA deficit, we performed 7×7=49 trials, varying the GABA projection parameter (y axis) and the postsynaptic weight parameter (x axis) independently through ranges of 0 to −45% and 0 to +60%, respectively. System response at 20, 30, and 40 Hz drive are shown. While it appears that joint increases in these parameters (i.e., a diagonal extending from the origin) show some preferential decrease in 40 Hz behavior, it is clear that no path through the 2D space is significantly schizophrenia-like. Dark green indicates areas in which there is minimal change (+/−5%) from control.

FIG. 5A-H. Response of schizophrenic model to 40 Hz drive.

Simulated EEG traces in response to 40 Hz drive for primary schizophrenic point (left panels), and secondary schizophrenic point (right panels), as defined in text. (A, B) Power spectra of primary and secondary schizophrenic points, respectively. (C, D) Model produced EEG traces. (E, F) EEG, averaged over two consecutive cycles. (G, H) Spiking histogram and firing rates for individual neuron subtypes. Averages over sets of two consecutive cycles are shown. X-axis label applies to all three histograms. Potent=potential; spks=spikes; CR=calretinin positive cells; PV=parvalbumin positive cells; PYR=pyramidal cells.

FIG. 6A-B. Simulated effects of phenyloin on control and schizophrenic models.

The therapeutic dose range for phenyloin is 10-20 mg/L, (40-80 μmol/L [62]). Lampl et al [63] and others [64], [65] have shown that phenyloin concentrations in this range produce a decrease in Na+ channel conductance of between 34% and 50%. Above, x axis indicates percent reduction in conductance of Na+ channel, and y axis indicates the power in given frequency band of the model when driven at 20, 30 and 40 Hz. Colored tick marks on right border of graphs indicate oscillatory behavior characteristic of control subjects (A) Schizophrenic model. When we implement virtual medication doses, by gradually decreasing gmax of the Na+ channel, no ameliorative effect (i.e., specific increase in 40 Hz activity) was seen. (B) Control model. There are no known clinical studies that are precisely comparable to the experimental paradigm we have used—that is, studies of control subjects receiving phenyloin at various doses, who receive auditory click train stimulation at 20, 30, and 40 Hz. However, studies that have looked at resting EEG activity at therapeutically relevant doses have shown that it tends to increase 20 Hz activity [64], and have inconsistent effects on frequencies in the 30 Hz range [66], [67]; it was not seen to have a significant gamma band effect. Also, laboratory experiments using kainite-induced gamma oscillations in hippocampal slice preparations showed that therapeutic levels of phenyloin (50 μM) had no effect on gamma oscillations (p=0.05) [68]. When applied to our control model at the above doses, we achieve similar effects.

FIG. 7A-B. Simulated effects of nifedipine on control and schizophrenic models.

This agent acts by blocking calcium channels. Electrophysiological studies have indicated that nifedipine can, depending on concentration, effectively decrease the slow inward Ca++ current by 50% or more. For illustrative purposes, we decreased calcium channel conductance by a maximum of 80%, in increments of 5%. Above, x axis indicates percent reduction in conductance of Ca+ channel, and y axis indicates oscillatory behavior (power in given frequency band) of model when driven at 20, 30 and 40 Hz. Colored tick marks on right border of graphs indicate oscillatory behavior characteristic of control subjects. (A) Schizophrenic model. When applied to the schizophrenic model, it did not show corrective effects, as expected. (B) Control model. For the 20 Hz range, clinical studies have shown no change under treatment with Ca++ channel blocker nimodipine [69], [70], or modest decreases in the relative power of this band [71]. For other frequency bands, general increases in resting EEG power [71] with treatment have been seen. Thus, simulation results are consistent with the clinical EEG literature.

FIG. 8. Ampakine application to schizophrenic model.

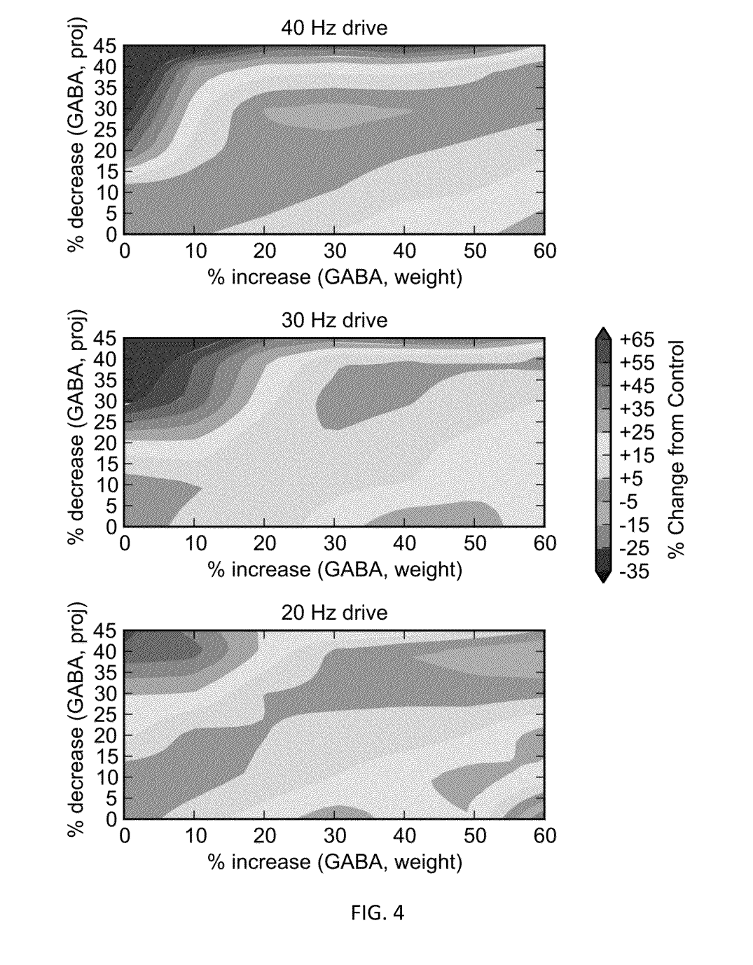

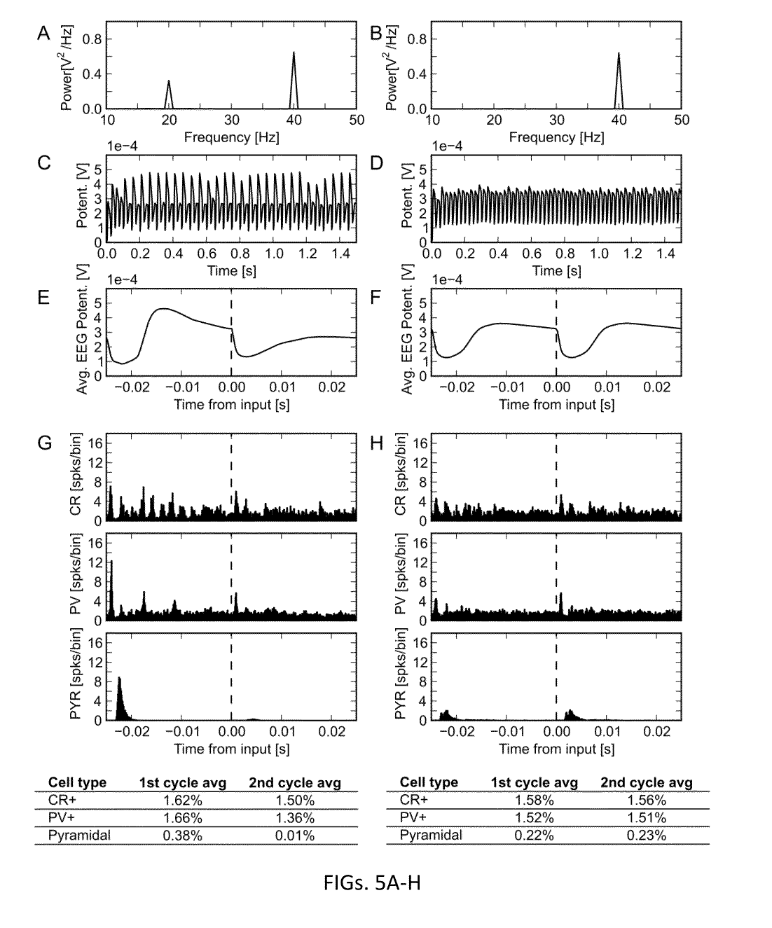

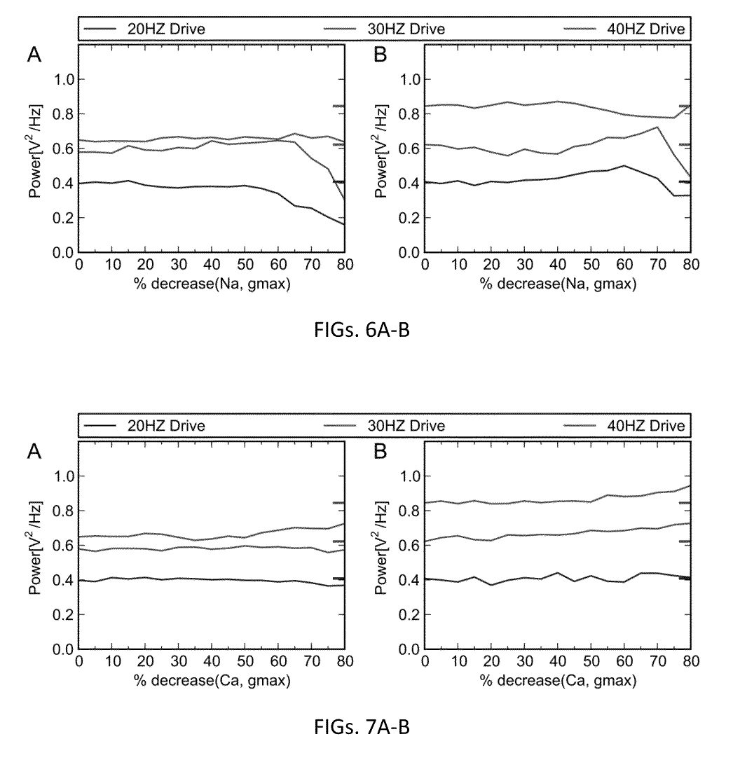

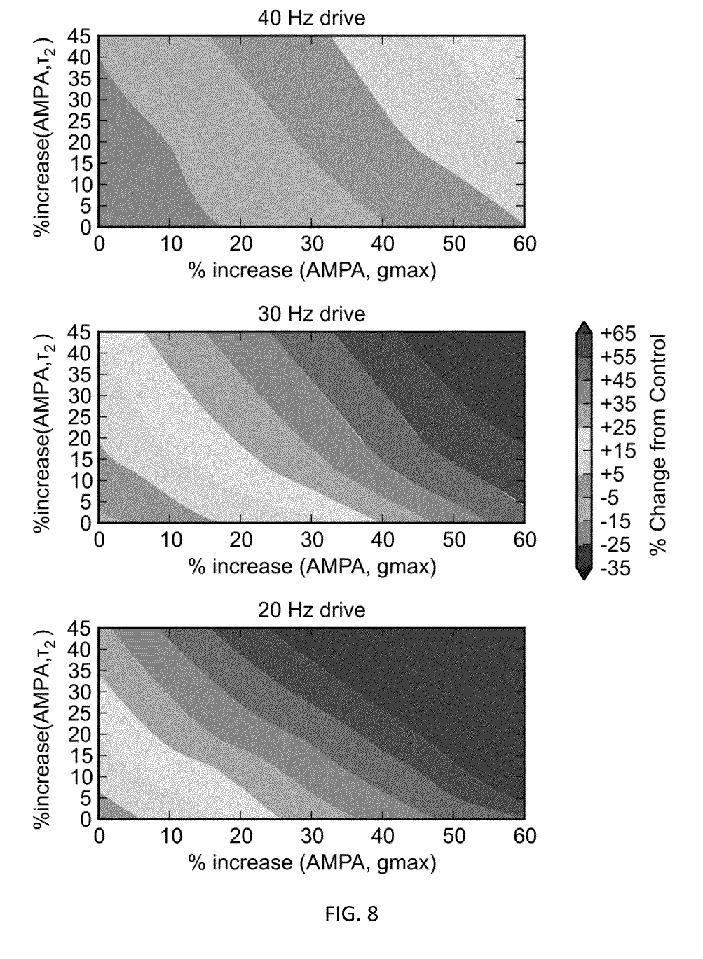

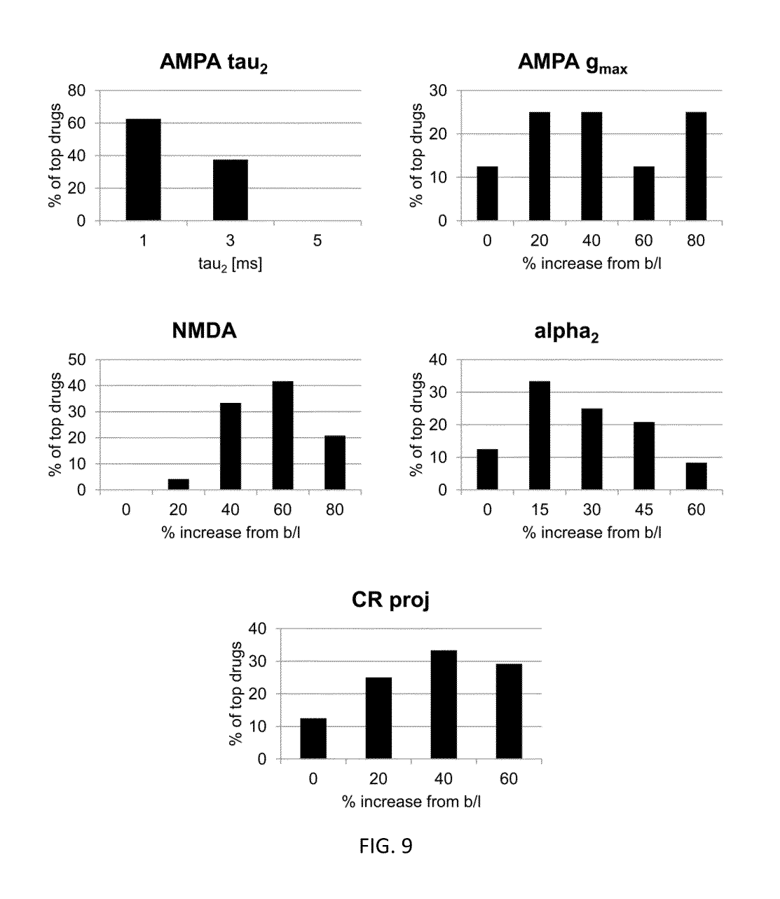

Ampakines act by increasing maximum conductance of the AMPA channel (denoted by gmax), increasing the decay time constant (denoted by τ2), or both. Moreover, various ampakines can differentially affect maximum amplitude and decay properties of the AMPA current [72]-[74]. To operationalize ampakine effects in model, we increased AMPA gmax by 0 to 60% (six gradations of 10%) and increased τ2 by 0 to 100% (five gradations of 20%), for a total of 30 iterations; we drove the model at 20, 30, and 40 Hz in each case. Color scale applies to all panels, and is identical to that of FIG. 4, to facilitate comparison. Here, % change refers to change from the unaffected case; therefore, 0 represents re-equilibration. From the figure, it is clear that there is no particular effect on 40 Hz activity—within a reasonable range of parameter assumptions, a virtual ampakine that effectively normalized 40 Hz resonance would create supraphysiologic levels of 30 and 20 Hz activity. This is consistent with clinical findings: On theoretical grounds, it was felt that this class of drugs may have an ameliorative effects on schizophrenia and, a number of ampakines have been developed for clinical use (CX516 [Ampalex], CX717, CX691/Org24448 [Faramptor], and LY451395) [75]. However, clinical trials [76] have not borne out their effectiveness in patient populations.

FIG. 9. Model response to simulated medication effects.

Breakdown, by mechanism of action, of top 24 most effective simulated drugs (those scoring 0.9 or higher). Y axis indicates fraction of top 24 drugs having the quantitative alteration shown for the given mechanism (e.g., slightly greater than 60% of the top drugs had an AMPA τ2 value of 1 ms). Titles correspond to mechanisms of action described in text. Baseline value for τMPA τ2 is 3 ms. b/l=baseline.

### DETAILED DESCRIPTION

Oscillatory brain activity is an emergent, system level behavior that stands at an intermediate level of complexity between the cellular and the clinical. A large amount of recent research has indicated that schizophrenic patients show synchronization deficiencies in neural processing [11], particularly in the gamma frequency band [12-17]. Importantly, there is also evidence that gamma activity subserves particular cognitive functions, such as perceptual binding within a particular sensory modality, or integration of information from different sensory modalities [18], to form a coherent percept. Thus, disturbed function may be etiologically related to some of the positive symptoms of schizophrenia, such as hallucinations or compromised reality testing.

Given the complexity of schizophrenia, it is unsurprising that computational modeling has been applied in an attempt to better understand this illness and its possible etiology. While there are exceptions [19,20], many have been abstract ANN (artificial neural network) style models, and they have tended to examine a single hypothetical pathology, such as connectivity disturbance [21], hyperdopaminergia [22], or deficient perforant path input into hippocampal formation [23]. One reason for this is that creating networks of biophysically detailed cellular models, and running large numbers of parameter assumptions (corresponding to different combinations of neural lesions or medication effects) are very computationally demanding undertakings. However, the development of computers with processing capacity several orders of magnitude greater than those of a generation ago now place us in a position such that we can begin to address these questions via “tissue level” computational work, and this is the approach we have taken here. Using a 72-processor supercomputer, the present inventors have created a biophysically detailed computational simulation of hippocampus, and use specific quantitative inability of the model to attune to 40 Hz stimulatory drive as a marker of the schizophrenic phenotype. Multiple putative schizophrenogenic cellular level abnormalities, as outlined above, were then introduced into the model in a graded and combinatorial way; the results showed that two distinct “clusters” of pathologies could recreate the schizophrenic phenotype. Then, virtual medication effects were applied to the schizophrenic model, and combinations that returned the model to its baseline (non-diseased) state were identified. The potentially ameliorative mechanisms identified are non-obvious, and do not represent simple reversals of the causative lesions.

Mechanistic Implications

While we acknowledge that 40 Hz oscillatory deficit is not a “classical” symptom of schizophrenia, we felt it was a highly appropriate outcome measure for this computational study for two reasons. First, given the likely importance of gamma band activity in subserving perceptual binding within and across sensory modalities and in cognition generally [18], and the core schizophrenic symptoms of compromised reality testing and hallucinations, a strong argument can be made that the gamma band biomarker is tapping into an important characteristic of the illness. Second, a growing body of clinical work suggests that it may represent an important endophenotypic marker of the disease. In a recent review of this literature [52], it was shown that across all frequencies (theta [4-7 Hz], alpha [8-12 Hz], beta [12-30 Hz], and gamma) and testing paradigms (steady state evoked potentials, induced responses, evoked responses, and resting state measurement), studies that showed the most robust and consistent evidence for a schizophrenic patient-specific phenomenon were those looking at steady state evoked responses in the gamma band. Because endophenotypes—as opposed to complex clinical phenotypes—may be more closely related to the genetic underpinnings of the disease, a focus on these markers may be extremely valuable in elucidating etiology and informing treatments [53].

Our modeling suggests that in the hippocampal etiology of schizophrenia, neural level abnormalities are perhaps not simply additive—that is, that more pathology, regardless of type, necessarily creates more illness. Rather, it appears that there may be one or more discrete combinations of abnormalities that give rise to the decreased gamma band activity that is associated with the illness. Of note, both sets of pathology we identified were characterized by co-occurring modest reduction in spine density, as well as reductions in NMDA functionality. Neither of these lesions alone, even occurring at extreme levels, was seen to be associated with schizophrenia-like model behavior. It appeared that particular combinations of GABA system lesions could lead to a specific, and modest, lessening of 40 Hz response; but no combinations of GABA lesions alone resulted in a pattern that was quantitatively similar to the schizophrenic dysfunction seen in the literature.

Significantly, the two clusters we identified were associated with dissimilar underlying neural dynamics. The most pronounced one (the primary point) showed a highly regular “beat skipping” quality, which created increased 20 Hz resonance in addition to decreased 40 Hz activity. A similar mechanism was seen in a previous modeling study [54]. This is particularly significant because that study implemented a very different mechanism—a lengthening of the decay time constant of the projections of PV+ interneurons—to generate similar behavior. This raises the possibility that this behavior may be an important mechanistic trait associated with the illness, at least in particular brain areas, and that different sets of cellular level abnormalities can give rise to it.

The other schizophrenic combination (the secondary point) we identified did not show this behavior, but appeared to arise from a dampening of 40 Hz behavior generally. This is consistent with the apparent inconsistencies in clinical research, in which some studies show a higher 20 Hz response [55], and many studies did not [17], [41] among schizophrenic patients. Schizophrenia's extreme heterogeneity has always been puzzling. This modeling work raises the possibility that different subtypes are associated with particular sets of neural abnormalities.

Post-mortem and other wet lab research methodologies tend to be very labor intensive, and investigating all combinations of possible neural abnormalities in large numbers of samples is not practical. The type of modeling study described here could be used as a guide, or hypothesis-generating tool, for laboratory research. Moreover, if clinical information is known about the tissue source (which is usually the case), the hypothesis that particular clusters of neural abnormalities correspond to particular subtypes can be tested.

Treatment Implications

Many traditional drug discovery efforts have involved identifying a cellular level abnormality associated with an illness, and creating an agent to counteract that particular deficiency. These efforts have not been entirely successful in the case of schizophrenia, and the modeling work here presents an alternative approach. As a test system, we used the primary point schizophrenia model, as described above. First, medications with no know antipsychotic efficacy were introduced to the schizophrenic model, to ensure that the model identified them as such. This is admittedly a “low bar”, but any test system to identify potentially effective agents should, at a minimum, be able to reject those that are clinically known to be inactive. Then, we carried out a series of 1,500 virtual medication trials, using five different potential drug mechanisms.

Perhaps the most surprising outcome of these simulated drug trials was the model's prediction that medications that decrease the decay time constant of the AMPA conductance would be potentially effective agents. This was apparent in looking at “wellness metric” data descriptively; it also emerged on two and three way analyses of variance. Of the five virtual drugs with highly significant p values, four involved an AMPA τ2 effect. This and other computational studies [54] have suggested that lingering or “blurring” of inhibitory processes may prevent the system from attuning to the relatively fast 40 Hz input drive. To the extent that a reduction of AMPA τ2 causes a sharpening of signaling, this could be beneficial.

Also, there were marked interactions between effects. Notably, the ANOVA showed a very weak single factor CR+ projection strength effect, but an extremely strong interactive effect with decreases in AMPA τ2 (the most robust interaction of all combinations tested). There was a very strong interaction between AMPA τ2 decrease and AMPA gmax as well. Increasing AMPA gmax and CR+ projection are similar in that alone, they would have the effect of increasing excitatory activity generally. It is therefore not surprising that ANOVA revealed these particular interactions—these combinations may result in an increased magnitude of a more “precise” signal.

The implications of these findings are threefold: First, it is possible that a given unsuccessfully tested mechanism (e.g., increasing AMPA conductance via ampakines) is not incorrect, strictly speaking, but rather incomplete—that is, in combination with other cellular level effects, amelioration of symptoms could be achieved. However, because of system complexity, it is difficult to determine a priori, based on deductive reasoning alone, which particular combination of “levers” could lead to a re-equilibration. Modeling can help identify the particular combination of mechanisms that will constitute effective medications.

Second, based on the existing literature, CR+ interneurons have not been implicated as a cause of schizophrenia [56]-[58], nor were they altered in our model to render it schizophrenic. The same is true of the decay time constant of the AMPA conductance. Nonetheless, altering these features helped to re-equilibrate the system, and return it to its control state. This implies that the search for effective antipsychotics need not be limited to neural elements that have been demonstrated in postmortem or other wet lab work to be abnormal in the illness.

Finally, this model makes specific, testable hypotheses. A drug that specifically increases the weight of CR+ post-synaptic projections or increases the conductance at these synapses, or that specifically decreases the APMA decay time constant may be efficacious for schizophrenia. In addition, treatment with combinations of agents that produce the effects shown in Table 7 are also expected to be efficacious.

Screening Methods

The methods described herein can be used to identify novel treatments for schizophrenia that produce one or more of: decreasing AMPA τ2, increasing CR+ projection strength, which can be used alone or in combination with each or with other treatments as described herein, e.g., treatments that increase AMPA conductance or increase NMDA activity.

Decreasing AMPA Decay Time Constant (τ2)

As noted above and demonstrated herein, lingering or “blurring” of inhibitory processes may prevent the neural system from attuning to the relatively fast 40 Hz input drive. A reduction of AMPA τ2 causes a sharpening of signaling, which is expected to be beneficial.

A number of methods known in the art can be used to identify compounds that decrease AMPA τ2, which is the decay time of the conductance (ion flow) through the AMPA channels in response to a stimulus. The stimulus can be anything that causes an activation of (that is, an opening and closing that allows ions to flow through) the AMPA channel, including but not limited to chemical (e.g., with AMPA or glutamate, or another agonist) and electrical stimulation. The stimulus is applied in the presence of a test compound. For example, a test cell that expresses functional AMPA receptors can be contacted with a test compounds, and τ2 can be measured in the presence of the test compound. That τ2 in the presence of the test compound is then compared to a reference τ2 that represents τ2 in the absence of the test compound. The reference τ2 can be determined in the same test cell (e.g., before or after application of the test compound) or in a control cell that is the same as the test cell. A compound that alters tau2 may alter the closing dynamics of the AMPA channel.

τ2 can be measured using any method known in the art. For example, τ2 can be measured electrophysiologically using patch clamp (e.g., whole cell, cell-attached, inside-out, or outside out patch clamp, optionally in a high-throughput setup, see, e.g., Arai et al., JPET 303:1075-1085, 2002; Arai et al., Mol Pharmacol 58:802-813, 2000; see also Zhao et al., Proc. IMechE Vol. 222 Part N: J. Nanoengineering and Nanosystems: JNN149; DOI: 10.1243/17403499JNN149; Clements et al., J Neurosci. 1998 Jan. 1; 18(1):119-27; Audinat et al., Neurochem Int. 1996 February; 28(2):119-36; and Jonas, E X S. 1993; 66:61-76); or two electrode voltage clamp, see, e.g., Wagner et al., Cell Physiol Biochem. 2000; 10(1-2):1-12.

Alternatively or in addition, the decay time constant τ2 can be measured by imaging, e.g., using methods of imaging intracellular calcium concentrations. A number of methods for imaging intracellular calcium concentrations ([Ca²⁺]ᵢ) are known in the art, and include contacting the test cell with a calcium imaging agent that emits a detectable signal depending on the [Ca²⁺]ᵢ, and detecting the signal, e.g., using an imaging method such as confocal or two-photon microscopy. See, e.g., Grienberger and Konnerth, Neuron. 2012 Mar. 8; 73(5):862-85. Calcium imaging agents include Oregon Green BAPTA-1, Calcium Green-1, Fura-2, Indo-1, Fluo-4, Rhod-2, and X-rhod-2, see Id.

The test cell (and control cell) can be any type of cell that is suitable for expression and measurement of AMPA τ2. For example, the cell can be a neuron, or can be a non-neuron, e.g., a mammalian cell transfected with or stably expressing the AMPA R. In some embodiments the cell in a xenopus oocyte. See, e.g., Wagner et al., Cell Physiol Biochem. 2000; 10(1-2):1-12. The test cell can be a whole cell or just part of a cell, e.g., isolated postsynaptic densities, see, e.g., Wyneken et al., Brain Res Brain Res Rev. 2004 December; 47(1-3):54-70.

In these embodiments, a test compound that decreases AMPA τ2 (as compared to the reference AMPA τ2, e.g., τ2 in a control cell in the absence of the test compound) is selected as a candidate therapeutic compound for the treatment of schizophrenia.

Increasing Weight of Calretinin-Positive (CR+) Post-Synaptic Projections

CR+GABAergic (gamma-aminobutyric acidergic) interneurons represent 10-30% of the total GABAergic cell population in the forebrain and appear to preferentially target other GABAergic cells in the neocortex. See, e.g., Caputi et al., Cerebral Cortex June 2009; 19:1345-1359. Test compounds that increase the synaptic weight of the CR+ neurons are expected to be beneficial in individuals with schizophrenia, as demonstrated herein.

A number of methods are known in the art for identifying test compounds that increase the synaptic weight of the CR+ neurons. For example, methods that evaluate the CR+ neurons within a network, e.g., in a brain slice (e.g., neocortical, allocortical, or hippocampal slices) or an in vitro network. Hippocampus was modeled computationally herein, and is technically considered to be allocortex, which is more primitive tissue. Neocortex is newer, evolutionarily, and is thought to subserve higher order functions (e.g., planning, judgment). The basic rules of neural connectivity are similar in hippocampus and neocortex (and other brain areas), so the effects found herein are broadly applicable.

CR+ neurons can be readily identified by the presence of calretinin expression; alternatively, CR+ neurons can be obtained from an animal expressing a fluorescent or other detectable marker under the control of a calretinin promoter, e.g., as described in Caputi et al., supra. Slice recordings can be made in the presence of a test compound, using methods known in the art, e.g., as described in Caputi et al., supra, wherein the CR+ neurons are stimulated (e.g., electrically or chemically stimulated) and the response in a postsynaptic cell, i.e., a neuron or interneuron measured. The postsynaptic cell can be inhibitory (interneurons) or excitatory (pyramidal cells).

The response can be compared to a reference response, e.g., the response to the same stimulus in the absence of the test compound.

In some embodiments, the response that is measured is long term potentiation at the postsynaptic synapse; short term potentiation (STP) or short term plasticity (Erickson et al., J Cogn Neurosci. 2010 November; 22(11):2530-40) measuring response to paired pulses; and measuring inhibitory postsynaptic current (IPSC) or inhibitory postsynaptic potential (IPSP) (Caputi et al., supra) in a postsynaptic cell, e.g., an inhibitory interneuron. Methods for recording and measuring IPSP, IPSC, and LTP are known in the art; see, e.g., Johnston and Wu, Foundations of Cellular Neurophysiology, MIT Press, 1995. For LTP see, e.g., Lamsa et al., Nature Neuroscience 8, 916-924 (2005). For measuring paired pulse, see Caputi et al., supra.

CR+ neuron axon terminals (the presynaptic side) form synaptic connections with their target cells (the postsynaptic side). GABA receptors, which are made up of multiple subunits of which there is a huge amount of heterogeneity in subunit composition, are expressed on the postsynaptic side. It is likely that those GABA receptors that are targets of CR+ neurons have a particular makeup. It has been shown that this is the case for other interneuron subtypes (e.g., parvalbumin+ interneurons, see Nyiri et al., European Journal of Neuroscience, Vol. 13, pp. 428±442, 2001). Thus, alternatively or in addition, the subtype of GABA A receptor subunits expressed in the CR+ neurons' target cells can be identified using methods known in the art, e.g., as described for parvalbumin+ neurons (see Nyiri et al., European Journal of Neuroscience, Vol. 13, pp. 428±442, 2001). This study looked at a different population of inhibitory interneurons—specifically, the parvalbumin (PV) positive cells, which exist in two morphologies (basket and chandelier), and also the cholecystokinin (CCK) neurons, having a basket morphology. Briefly, this paper shows that these interneuron subtypes differ in terms of the subunit composition of their postsynaptic GABA receptors. In addition, Olsen and Sieghart, Pharmacol Rev 60:243-260, 2008, summarizes a number of ways in which GABAA subtypes can be identified, e.g., genetically (see Table 1 (p. 247)). Using these methods or others known in the art, the subunit composition of GABAA synapses that receive CR+ projections can be determined. Mohler et al., JPET 300:2-8, 2002. Once the subunit composition of the GABAA receptors on the synapses that receive CR+ projections is known, a compound that targets that specific combination can be selected. A significant number of compounds have now been developed that display GABAA receptor subtype selectivity by affinity or efficacy, or by both (see Table 1 of Rudolph and Knoflach, Nature Reviews Drug Discovery 10:685-697, 2011). Alternatively, to identify a drug that targets the specific subunit combination, the subunits can be expressed recombinantly, e.g., in mammalian cells and test compounds can be applied to identify those compounds that effectively increase the synaptic weight of the CR+ neurons (e.g., by increasing the conductance (e.g., increasing amplitude or decay time) of those postsynaptic GABAA receptors, which can be measured in single or cultured cells, or by measuring LTP, STP, IPSP, IPSC, or paired pulse as described above in networks, e.g., in slice or in vitro networks). See, e.g., Rudolph and Knoflach, Nature Reviews Drug Discovery 10:685-697, 2011

In these embodiments, a test compound that increases the synaptic weight of the CR+ neurons is selected as a candidate therapeutic compound for the treatment of schizophrenia.

Test Compounds

Included herein are methods for screening test compounds, e.g., polypeptides, polynucleotides, inorganic or organic large or small molecule test compounds, to identify agents useful in the treatment of schizophrenia.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, the test cell or sample is, or is derived from (e.g., a sample taken from) an in vivo model of schizophrenia. For example, an animal model, e.g., a rodent such as a rat, can be used.

A test compound that has been screened by a method described herein and determined to decrease AMPA τ2 or increase CR+ projection strength can be considered a candidate compound. A candidate compound that has been screened, e.g., in an in vivo or in vitro model of schizophrenia and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that decrease AMPA τ2 or increase CR+ projection strength) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating schizophrenia. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of schizophrenia, e.g., an animal model as described in Jones et al., Br J. Pharmacol. 2011 October; 164(4): 1162-1194. The animal can be monitored for a change in the disorder, e.g., for an improvement in a parameter of the disorder, e.g., a parameter related to clinical outcome. In some embodiments, the parameter is a positive symptom and an improvement would be a reduction in the severity, duration, or frequency of the positive symptom. In some embodiments, the subject is a human or primate, e.g., a human with schizophrenia, and the parameter is psychosis. See, e.g., Jones et al., supra.

In some embodiments, the methods can include the use of a computational model as described herein to predict an effect of a compound on schizophrenia in a subject, e.g., as described in U.S. Pat. No. 7,945,392, which is incorporated by reference herein in its entirety.

Once identified, the candidate therapeutic compound can be formulated for use a a therapeutic compound in a pharmaceutical composition. Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration. Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY).

Combination Therapies

Also described herein are methods of treating schizophrenia that include administration of combinations of known drugs that provide improved responses in the model described herein. For example, a combination of an NMDA agonist and a GABAA-alpha 2 agonist can be administered. A number of NMDA agonists are known in the art, e.g., UBP646, UBP512, UBP551, CIQ, Glycine, D-cycloserine, glycine type I (GlyT1) transporter inhibitors, and D-serine (see Tuominen et al., Schizophrenia Research 72 (2005) 225-234 and Monaghan et al., Neurochem Int. 2012 September; 61(4):581-92). Glycine is a co-agonist at the NMDA receptor; the glycine type I transporter serves to clear glycine, and thus lower glycine levels in the synaptic cleft area. Therefore, GlyT1 inhibitors are effectively NMDA agonists. Glycine type I (GlyT1) transporter inhibitors include, e.g., sarcosine (N-methylglycine) or RG1678 (see Javitt et al., Handb Exp Pharmacol, 2012; (213):367-99).

GABAA-alpha2 agonists include MK-0777 (also known as TPA-023), TPA023B and MRK-409 (MK-0343), which specifically act at the α2 and α3 GABAA subtypes), see, e.g., Rudolph and Knoflach, Nature Reviews Drug Discovery 10:685-697, 2011.

Alternatively or in addition, the methods can include the administration of a combination of an NMDA agonist and an AMPAkine NMDA agonists as described above. AMPAkines include CX516, CX717, CX691 (faramptor), LY451395 or CX546 (see Arai and Kessler, Curr Drug Targets. 2007 May; 8(5):583-602; Grove et al., J Med Chem. 2010 Oct. 28; 53(20):7271-9). Piracetam and aniracetm are know to be positive allosteric modulators of the AMPA channel.

Dosage

An “effective amount” of a compound administered according to a method described herein is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims. The first example below illustrates the network model's ability to attune to 20, 30, and 40 Hz stimulation in the baseline condition. Subsequent examples show the results of implementation of schizophrenogenic cellular lesions, and the results of trials that incorporate the effect of both known medications and virtual antipsychotic drugs.

### Methods

The following methods were used in the Examples set forth below.

Computational Model

The hippocampal model consists of 160 pyramidal cells and interneurons of three subtypes—30 basket cells, 30 chandelier cells, and 20 calretinin-positive (CR+), or interneuron projecting, cells. For pyramidal cells, we used the 64 compartment model described by Traub et al [24]. Interneuron models were based on the 46 compartment model of Traub and Miles [25]. Both include realistic dendritic arbors and incorporate Na⁺, Ca⁺, K_{DR}⁺, K_{AHP}⁺, K_{C}⁺ and K_{A}⁺ channels with Hodgkin-Huxley dynamics distributed along the somato-dendritic axis. Interneurons of different subclasses were defined by their axonal projections patterns, based the hippocampal model described by the author [26]. Full details of individual neuron models and their connectivity, a description of the manner in which a simulated EEG was calculated, and details of the model's hardware implementation were as follows.

Individual Neuron Models

The hippocampal model consists of a total of 240 simulated neurons: 160 pyramidal cells, and interneurons of three subtypes (30 basket cells, 30 chandelier cells, and 20 calretinin-positive [CR+], or interneuron projecting, cells). For pyramidal cells and interneurons, we used the 64 compartment model described by Traub et al (Journal of Physiology (London) 481: 79-95 (1994)), and the 46 compartment model of Traub and Miles (Journal of Computational Neuroscience 2: 291-298 (1995)), respectively.

Each compartment of the individual neuron models is represented as follows:

$${C}_{m} \frac{d {V}_{m}}{d t} = \frac{( {E}_{m} - {V}_{m} )}{{R}_{m}} + \frac{( {V}_{m}^{′} - {V}_{m} )}{{R}_{a}} + {I}_{syn} + {I}_{ionic} ,$$

where Vₘ is the transmembrane potential and V′ₘ is the transmembrane potential of the adjacent compartment, Cₘ is the membrane capacitance, Eₘ is the resting membrane potential, Rₘ is the membrane resistance, Rₐ is the axial resistance, and I_{syn} and I_{ionic} terms are the sums of current from voltage-dependent ionic channels and synaptic channels, respectively; values for the constants used in the neuron models are shown in Tables 1 and 2.

TABLE 1
Summary of compartmental parameters for neuronal models.

| Parameter | Definition | Value |
|---|---|---|
| Eₘ | Resting membrane potential | −60.0 mV |
| C_{M} | Specific membrane capacitance | Vary by |
| R_{M} | Specific membrane resistance | compartment and |
| R_{A} | Specific axial resistance | cell type |
| | | (see below). |
| Rₐ | Axial resistance | Rₐ = 4lR_{A}/(πd²) |
| Cₘ | Membrane capacitance | Cₐ = πldC_{M} |
| Rₘ | Membrane resistance | Rₐ = R_{M}/(πld²) |

TABLE 2
Model parameters by cell type and subcellular location.

| | Pyramidal Cells | | | Interneurons | | |
|---|---|---|---|---|---|---|
| | | Axonal | | | Axonal | |
| | | Initial | | | Initial | |
| Parameter | Soma | Segment | Dendrite | Soma | Segment | Dendrite |
| C_{M} | 0.75 | 0.75 | 1.5 | 0.75 | 0.75 | 0.75 |
| [μF/cm²] | | | | | | |
| R_{M} | 50 | 1.0 | 25 | 50 | 1.0 | 50 |
| [KΩcm²] | | | | | | |
| R_{A} | 0.2 | 0.1 | 0.2 | 0.2 | 0.1 | 0.2 |
| [KΩcm] | | | | | | |

The individual ionic channel currents take the form

I_{channel}=G_{channel}(E_{channel}−Vₘ), (2)

where I_{channel} is the current of an ionic channel, E_{channel} is the reversal potential of that channel, and G_{channel} is the variable conductance of that channel. Conductances in this model are those used in the aforementioned articles (Traub et al., Journal of Physiology (London) 481: 79-95 (1994), and Traub and Miles Journal of Computational Neuroscience 2: 291-298 (1995)).

Synaptic currents were also included. AMPA and GABA_{A} currents take the form of Equation 3, where the reversal potential is 0 mV for NMDA, 45 mV for AMPA and −82 mV for GABA. Conductances were assumed to obey a dual exponential function, as follows:

$${G}_{syn} ( t ) = H ( t - {t}_{n} ) \frac{W {Ag}_{max}}{{τ}_{1} - {τ}_{2}} ( {e}^{- ( t - {t}_{n} ) / {τ}_{1}} - {e}^{- ( t - {t}_{n} ) / {τ}_{2}} ) , for {τ}_{1} > {τ}_{2} , \qquad ( 3 )$$

where H(t−tₙ) is the Heaviside function, A is a normalization factor such that the maximum conductance is gₘₐₓ, τ₁ and τ₂ are time constants, and W is the weight factor for the particular connection. The NMDA channel takes the form shown in Equation 3, with modifications to instantiate magnesium block behavior (Zador et al., Proceedings of the National Academy of Sciences of the USA 10: 6718-6722 (1990). All parameters for synaptic connections are given in Table 3.

TABLE 3
Synaptic channel parameters.

| Parameter | Definition | Value |
|---|---|---|
| g_{max, NMDA} | Maximum conductance of NMDA | 160 × 10⁻¹² S |
| g_{max, GABA}_{_{A}} | Maximum conductance of GABA_{A} | 40 × 10⁻¹² S |
| g_{max, AMPA} | Maximum conductance of AMPA | 80 × 10⁻¹² S |
| τ_{1, NMDA} | First time constant of NMDA | 0.080 S |
| τ_{2, NMDA} | Second time constant of NMDA | 0.000670 S |
| τ_{1, GABA}_{_{A}} | First time constant of GABA | 0.003 S |
| τ_{2, GABA}_{_{A}} | Second time constant of GABA | 0.008 S |
| τ_{1, AMPA} | Fist time constant of AMPA | 0.003 S |
| τ_{2, AMPA} | Second time constant of AMPA | 0.003 S |

Connectivity and Stimulation

Cell to cell connectivity was based on hippocampal modeling previously described by the author (Siekmeier, Behavioural Brain Research 200: 220-231 (2009)). In summary, the following assumptions were made: (1) Pyramidal cells (PCs) project very sparsely to other pyramidal cells. (2) Basket cells densely innervate somata and proximal dendrites of PCs. (3) Chandelier cells synapse only on axonal initial segments of PCs. (4) Calretinin cells project densely to other interneurons; they do not innervate PCs. All connectivity parameters are presented in Table 4.

For pyramidal projections, postsynaptic receptors were divided between AMPA and NMDA synapses. Based on an extensive review of the hippocampal neuroanatomy literature, PC to PC projections were taken to be 10% NMDA and 90% AMPA (Nicholson et al., Neuron 50: 431-442 (2006); Nusser., Current Opinion in Neurobiology 10: 337-341 (2000)), and PC to interneuron projections were 40% NMDA and 60% AMPA (Sah et al., Journal of Physiology 430: 605-616 (1990); Baude et al., Neuroscience 69: 1031-1055 (1995); Nyiri et al., Neuroscience 119: 347-363 (2003)). All interneuron projections formed GABA synapses.

The model was driven by spike train at the given test frequency (20, 30, or 40 Hz). The drive was delivered to all pyramidal cells with a dedicated AMPA-type receptor, and 50% of interneurons. This was based on anatomical data which indicates that incoming projections impinge on relatively fewer interneurons, compared with PCs (Binzegger et al., Journal of Neuroscience 24: 8441-8453 (2004)).

In the context of the entire hippocampal formation, our model represents a very small piece of tissue; thus, the amount of innervation received by a given cell is considerably smaller than that received by a hippocampal cell in vivo. For example, actual CA1 PCs receive about 30,000 excitatory and 1,700 inhibitory inputs (Megias et al., Neuroscience 102: 527-540 (2001); the inputs received by the cells in our model were orders of magnitude lower than this. To compensate, we multiplied the synapses of the model by a weight factor of 10. Also, a given basket cell projects to a PC and forms a dense nexus of connections around the receiving cell's soma and proximal dendrites. Similarly, chandelier cells project densely to IS, and because of this, have a particular pronounced effect on PC output. Weight multipliers for these two cell types are increased to reflect these facts, as indicated in Table 4. For model input drive, the multiplier was 500.

TABLE 4
Model connectivity. Model was connected randomly using the indicated
probabilities for each connection type.

| | Number | | Post-Synaptic | Connectivity | Connection |
|---|---|---|---|---|---|
| Cell type | of Cells | Post Synaptic target | receptors | probability | Weights |
| Pyramidal | 160 | Pyramidal dendrites | NMDA and | 5% | 10 |
| | | Basket dendrites | AMPA | 21% | 10 |
| | | Chandler dendrites | | 21% | 10 |
| | | CR+ dendrites | | 27% | 10 |
| Basket | 30 | Pyramidal proximal dendrites and soma | GABA | (13%)* | 60 |
| Chandler | 30 | Pyramidal IS | GABA | (13%)* | 120 |
| CR | 20 | Basket dendrites | GABA | 100% | 30 |
| | | Chandler dendrites | | 100% | 10 |
| | | CR+ dendrites | | 100% | 30 |

* Pyramidal cells received projections from 13% of basket cells and 13% of chandelier cells.

Simulated EEG

To compare the behavior of our system to the experimental data, it was necessary to calculate a simulated EEG for the system. Biologically, an EEG signal is produced by current flowing through ion and synaptic channels, thus creating a changing electric potential measured at the scalp. According to the Nun\tilde{e}z equation (Electric fields of the brain: The neurophysics of EEG. New York: Oxford University Press. (1981) 484 p), this potential is found by

$$Φ ( \vec{r} , t ) = \frac{1}{4 π σ} {\sum }_{n = 1}^{N} \frac{{I}_{n} ( t )}{{R}_{n}} , \qquad ( 4 )$$

where Φ is the potential measured at a point \vec{r} at time, t, and Rᵢ is the distance, through a medium with conductivity, σ between the current, Iₙ and the point (the electrode).

For the simulation, we assumed that the tissue modeled was of negligible spatial distribution; distance R is assumed to be 6.8 mm, the average skull thickness (Li et al., International Journal of Vehicle Safety 2: 345-367 (2007)). We used an estimated median of value for conductivity of the brain of 0.251 S/m (Huang et al., Neuroimage 37: 731-748 (2007)). As in previous studies, we considered only the contribution of the excitatory channels, as this is felt to be the main contributor to EEG activity. Therefore, the EEG is calculated in our study by

$$Φ ( t ) = \frac{1}{4 π σ R} {\sum }_{n = 1}^{N} {I}_{n} , \qquad ( 5 )$$

Where Φ is the potential measured at a single point outside of the skull, and N is the total number of excitatory synaptic channels.

Computing Details

The models described above were implemented using the General Neural Simulation System (GENESIS) version 2.3 (Bower and Beeman, (1998) The Book of GENESIS: Exploring Realistic Neural Models with the GEneral NEural SImulation System. Santa Clara, Calif.: Springer-VerlagTelos (1988)), using programs written in C++ with MPI to conduct parameter searches. The backward Euler integration method, as implemented in standard GENESIS, was used to solve the above equations with a 0.1 ms time step. All modeling was carried out on a 72-processor dedicated Beowulf computer cluster (PSSC Labs/Professional Service Supercomputers, Lake Forest, Calif.), running under RHEL5 64-bit Linux operating system within the Laboratory for Computational Neuroscience at McLean Hospital.

Implementation of Putative Schizophrenogenic Cellular Level Abnormalities

Glutamatergic System Dysfunction:

A number of recent research studies have found decreased density of NMDA synapses in schizophrenic hippocampus and/or hypofunction of these synapses, and have quantified this effect. Tsai et al [27], in postmortem work, found a decrease of 37% in glutamate levels in hippocampus of schizophrenics. This group also found a 55% increase in N-acetylaspartylglutamic acid (NAAG) in this area; together with work by Bergeron et al [28] that demonstrated an inverse relationship between NAAG level and NMDA current, this suggests that schizophrenic patients may experience NMDA hypofunction via decreases in the conductance of the NMDA channel. Law and Deakin [29] found a decrease in the obligatory NMDAR1 subunit of the NMDA receptor of 40%. Similarly, Harrison et al [30], in postmortem work looking at markers of glutamate receptors in schizophrenic hippocampus, found a decrease of 26% in mRNA coding for NR1, a subunit of the NMDA receptor.

Thus, the research suggests a spectrum of possible NMDA deficits. To capture the full range of possible values, we implemented NMDA effects by decreasing maximum conductance (gmax) of the model NMDA receptors by 0 to 45%, in increments of 5%. We also performed trials in which the number of NMDA receptors was decreased through this range, and found quantitatively similar effects.

Connectivity Disturbances:

A “pruning hypothesis” of schizophrenia has long been suggested [9], [31]. Much of the substantiation for this, however, came from indirect measurements (e.g., decreased neuropil volume). Studies have examined this quantitatively by looking at density of spines on neuronal dendrites. Law et al [32] found a decrease in levels of mRNA for spinophilin (a marker for dendritic spines) of 44.5%, on average. Garey et al [33], in a postmortem study Golgi staining, saw a decreased spine density in temporal lobe of patients of 59.4%. DeVito et al [34], using a genetic knockout model of NMDA receptor hypofunction (a serine racemase knockout mouse), found a decreased dendritic spine density of 40.5%. To capture the full range of possible values, we decreased pyramidal cell spine density from 0 to 60%, in increments of 5%.

GABA System Dysregulation:

Heckers et al [35] found decreased expression of mRNA for two isoforms of the GABA-synthesizing enzyme glutamic acid decarboxylase GAD65 and GAD67 decreased by 14% and 4%, respectively, in schizophrenic hippocampi. Bird et al [36], in a postmortem study of brains of psychotic patients found GAD to be decreased by 48.2%. Fatemi et al [37] and Torrey et al [38] found decreases in reelin in schizophrenic hippocampus of 29% and 46%, respectively. However, other studies have found increases in GABA receptor binding. For example, Benes et al [39] showed increases of 45% to 82% depending on subfield of hippocampus. It is felt that this may represent a compensatory upregulation of postsynaptic GABA receptors, in response to decreased GABAergic activity [40].

To apply these changes, the decrease in GABAergic tone was simulated by decreasing the number of GABAergic projections (that is, projections from model interneurons) from 0 to 45% in increments of 7.5%; the increased weight of postsynaptic GABA receptors was simulated by increasing the weight factor at these synapses from 0 to 60%. These changes were made in tandem, testing 7 “ordered pairs” of parameter values ([0, 0] to [−45%, +60%,]), where each element was [change in GABAergic tone, GABA postsynaptic weight change]. This was done because when searching large parameter spaces, adding an additional dimension increases the number of trials multiplicatively, and searching four dimensions for the current problem would have been prohibitively time consuming.

Calculation of Illness Metric

A metric was created to quantify the “schizophrenic-ness” of a given model run, based on its quantitative similarity to the experimental findings of Teale et al [16]. This study was used because it employs a steady state evoked potential (SSEP) task, and detailed source localization carried out in the study revealed that the source of the oscillatory activity recorded was temporal lobes, and thus may be hippocampal in origin. Based on their data (their FIG. 6, p. 1486), which shows 40 Hz oscillatory activity as a function of time when patients are receiving the stimulus, at maximum patients showed a decrease of approximately 26% at this frequency, compared with controls (this represents an average over left and right hemispheres). The many SSEP experiments that have been carried out on schizophrenic patients indicate that when exposed to 20 or 30 Hz stimulation, patients did not show a response significantly different from controls [41].

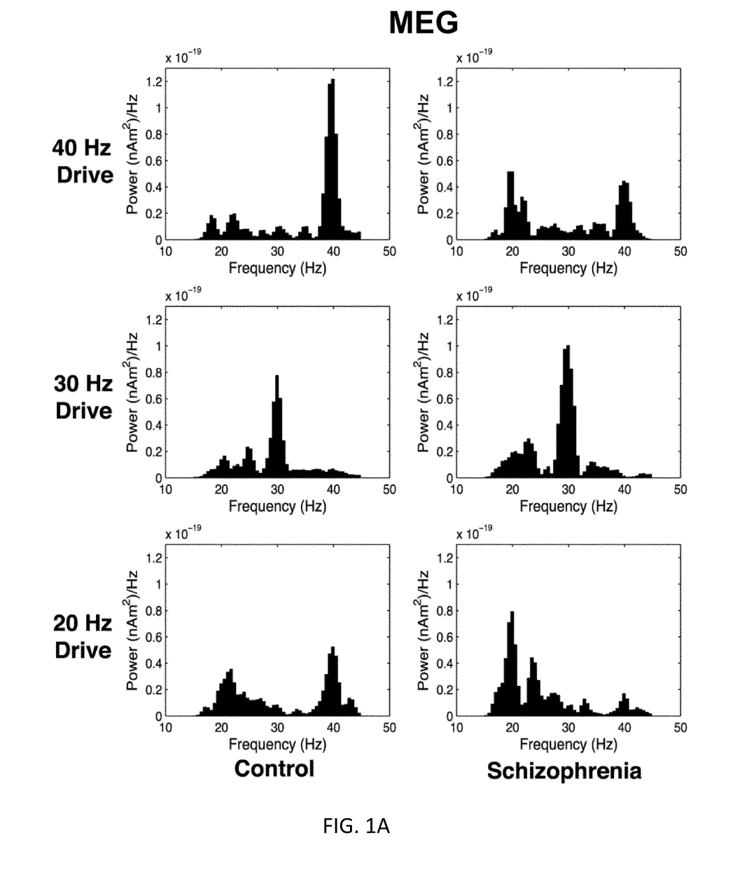

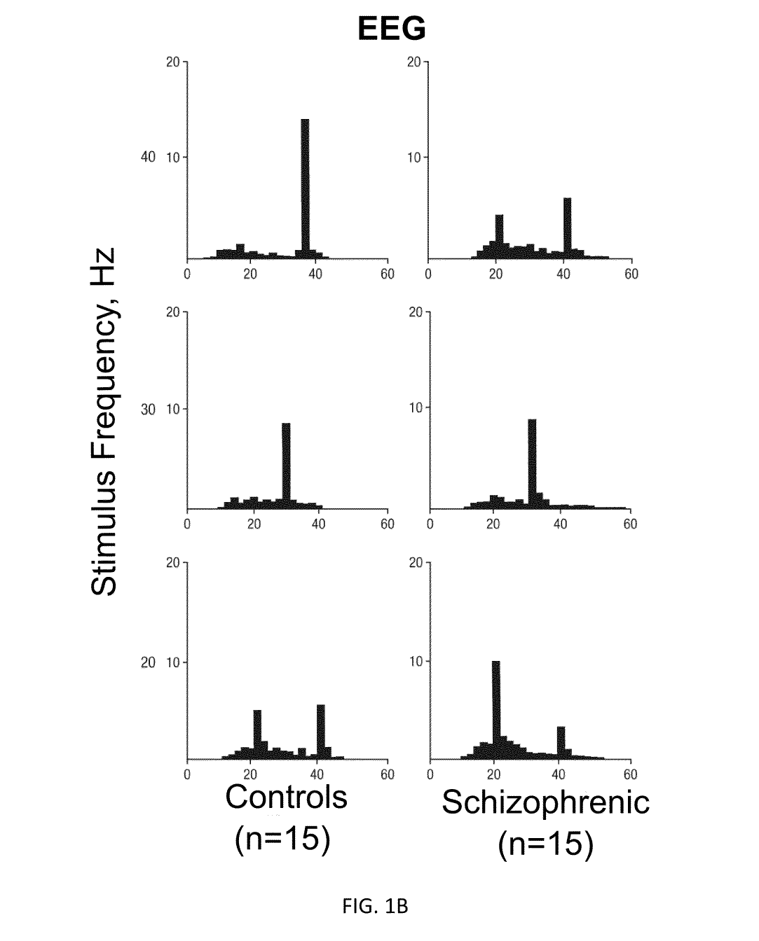

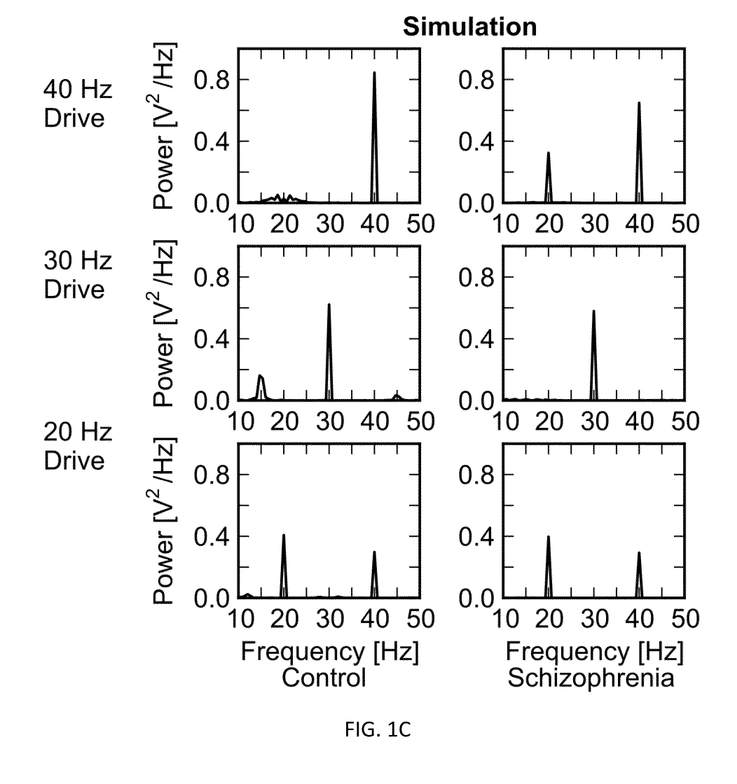

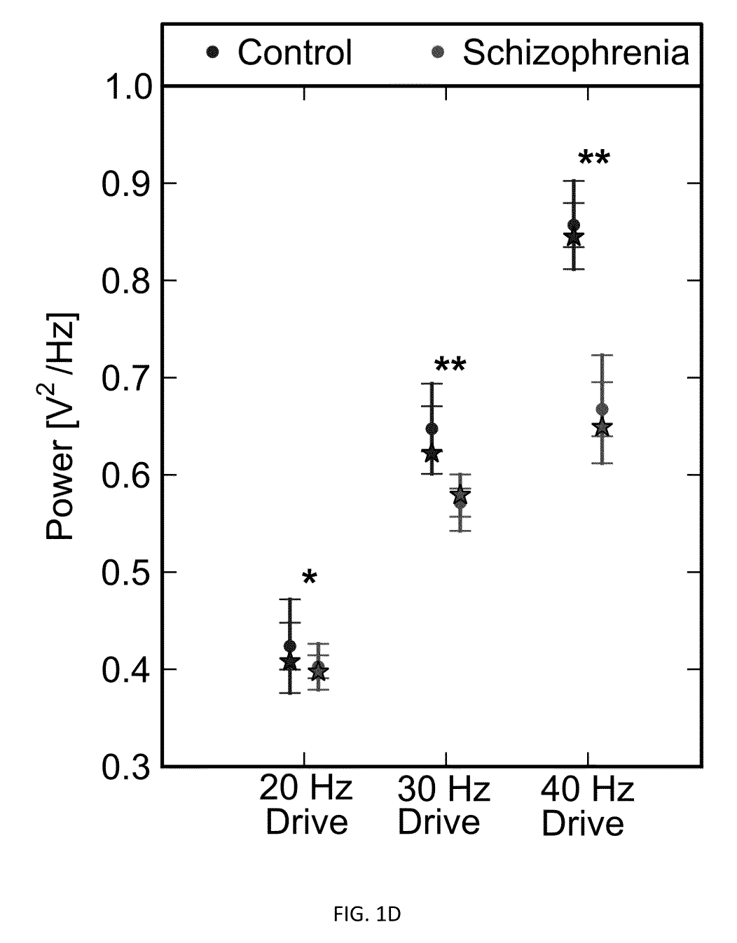

Therefore, for a model to said to be schizophrenic: (1) 20 and 30 Hz activity were required to be within a given tolerance of the baseline case. We used +/−7.5% for this value, based on the standard deviation of our 20 simulated control patients (FIG. 1); models that failed for either frequency were given a score of 0. (2) 40 Hz activity was required to be significantly decreased from the baseline condition. To quantify this, percentage decrease of schizophrenic condition vs. control condition was calculated (that is, [power of 40 Hz response, baseline condition]−[power of 40 Hz response, schizophrenic condition]/[power of 40 Hz response, baseline condition]). If this equaled 26%, the model received a score of 1; to the extent that this differed from 26%, in absolute value, the score was decreased. Calculation of the illness metric was performed as follows.

We created an “illness metric” as a quantitative definition of schizophrenia for this study, based on the driven frequency abnormalities observed in the disease. The metric varies from 0 to 1 with 1 being the most schizophrenic-like. The metric is defined as:

$$I ( q ) = {\prod }_{f = 20 , 30 , 40} {I}_{f} ( {ρ}_{f} ( q ) ) , \qquad ( 6 )$$

where I(q) is the illness metric at the point q in the parameter space, l_{f} is the individual illness metric at a drive frequency of, f, and ρ_{f} is the height of power spectrum peak at that frequency. The individual frequency metrics measure the contribution of each frequency to the final metric. For drive frequencies of 20 Hz and 30 Hz, this metric is simply a pass or fail test. Specifically:

$${I}_{f} ( {ρ}_{f} ) = \{ \begin{matrix}1 & if P ( {S}_{f} ) - {δ}_{2 , f} \leq P ( {ρ}_{f} ) \leq P ( {S}_{f} ) + {δ}_{I , f} \\ 0 & otherwise\end{matrix} \} , \qquad ( 7 )$$

where P(S_{f}) is the percent change from control of the ideal schizophrenic power and δ_{1,f} and δ_{2,f} are arbitrary constants used to describe the acceptable range for each frequency. This gating effect for 20 and 30 Hz simply eliminates all points which fall in a non-biologically realistic range. For the 40 Hz drive frequency, a graded function is used to rank the effects. This emphasizes the important role that gamma band oscillations are thought to play in schizophrenia. The function used for the 40 Hz drive frequency is:

$${I}_{40} ( {ρ}_{40} ) = \{ \begin{matrix}0 & if P ( {ρ}_{40} ) < P ( {S}_{40} ) - {δ}_{2 , 40} \\ 1 - \frac{( 1 - a )}{{δ}_{2 , 40}} ( P ( {ρ}_{40} ) - P ( {S}_{40} ) ) & if P ( {S}_{40} ) - {δ}_{2 , 40} \leq P ( {ρ}_{40} ) < P ( {S}_{40} ) \\ 1 + \frac{( 1 - a )}{{δ}_{1 , 40}} ( P ( {ρ}_{40} ) - P ( {S}_{40} ) ) & if P ( {S}_{40} ) \leq P ( {ρ}_{40} ) \leq P ( {S}_{40} ) + {δ}_{1 , 40} \\ 0 & if P ( {ρ}_{40} ) > P ( {S}_{40} ) + {δ}_{2 , 40}\end{matrix} \} \qquad ( 8 )$$

where a is the lowest score possible for a peak within bounds (0.01). This metric is controlled by several constants, as shown in Table 5.

TABLE 5
Illness metric parameters.

| Drive Frequency[Hz] | δ₁ | δ₂ | P(S_{ƒ}) |
|---|---|---|---|
| 20 | 0.075 | 0.075 | 0.00 |
| 30 | 0.075 | 0.075 | 0.00 |
| 40 | 0.26 | 0.26 | −0.26 |

To ensure that the most highly schizophrenic case identified represented a robust model behavior, 20 simulated schizophrenic subjects and 20 control subjects were created. In order to create a simulated subject, the random number generator was re-seeded to generate a new model, and it performed the experimental task. Thus, each simulated subject had different specific cell-to-cell pattern of connectivity, but the projection probabilities between cell types (as defined in Table 4) were identical. Using these data, we ran a mixed model ANOVA, entering Group (control, schizophrenic) as a between subjects factor and Frequency (20 Hz, 30 Hz, 40 Hz) as a repeated measures factor. To test the specificity of putative ANOVA findings, hierarchical regressions were run.

### Example 1

### Reproduction of Baseline Oscillatory Activity

After tuning, we drove the hippocampal model at 20, 30, and 40 Hz; a simulated EEG was generated for each and was analyzed via fast Fourier transform (FFT) to determine which frequencies were present. The model reproduced, in a quantitatively similar way, frequency behaviors shown in control subjects (FIG. 1A [left panels] and 1B [left panels] experimental; FIG. 1C [left panels] model output).

To confirm these model results, 20 simulated control subjects were created as described in the Methods section. The results of these runs are shown in FIG. 1D (blue points). It is clear that the behavior of our simulated index control subject is representative of the group of simulated controls, and that this group is similar to that of the control subjects of experimental studies.

### Example 2

### Effects of Putative Schizophrenogenic Cellular Level Abnormalities on Model Behavior

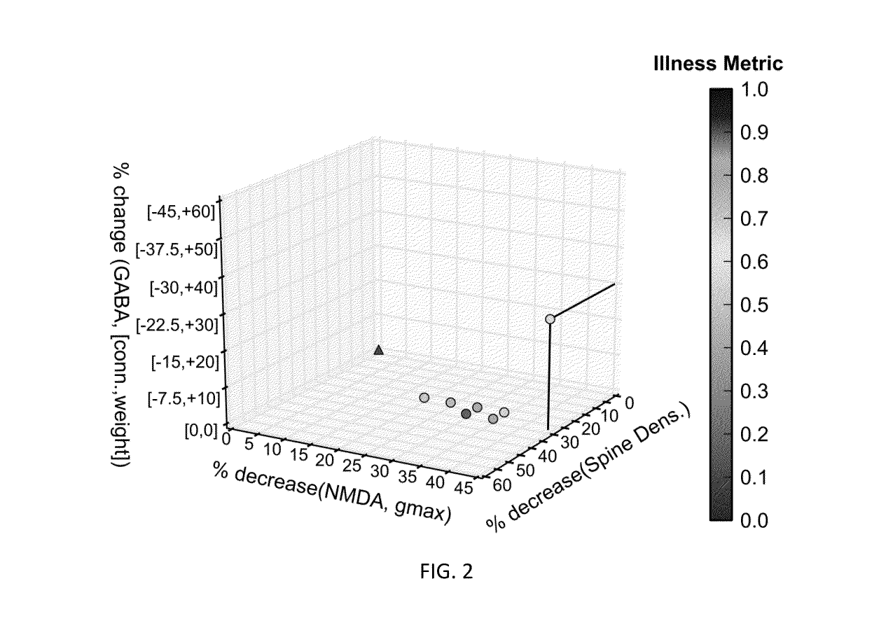

The manner in which the cellular level pathology that has been observed in schizophrenic hippocampus was instantiated as parameter changes in the model is detailed in Methods. Briefly, decreased NMDA activity was operationalized by decreasing maximum conductance (gmax) of the model NMDA receptors (in 10 increments); connectivity deficits were operationalized by decreasing pyramidal cell dendritic spine density (13 increments); and GABA system dysregulation was implemented by a joint decrease in GABA tone and increase in postsynaptic weight (7 increments). Iterations representing all possible levels of the aforementioned cellular level lesions were run: that is, we exhaustively searched the parameter space, running 10×13×7=910 iterations in total. Each iteration consisted of three trials; in each, the network was driven at a given frequency (20, 30, or 40 Hz), and a simulated EEG was written to file and was analyzed via fast Fourier transform (FFT) to determine which frequencies were present, and their relative power. The degree to which this matched the pattern seen in the clinical studies (i.e., the degree to which there was a specific deficit in 40 Hz response) was quantified using the illness metric, which ranged from 1 (most schizophrenic) to 0, as described in Methods. FIG. 2 graphically depicts the results of these trials.

Clearly, a number of points produce schizophrenia-like results. There is a prominent cluster centered at a point characterized by an NMDA decrease of 30%, a spine density decrease of 30%, and a GABA deficit of 0 (which we will call the “primary point”). There is another point characterized by an NMDA decrease of 45%, a spine density decrease of 30%, and a GABA system defect of (−37.5, +30%), as defined in Methods (which we will call the “secondary point”). For the primary point, power spectra of oscillatory activity in response to 20, 30, and 40 Hz drive is shown in FIG. 1C, in comparison with control behavior (FIGS. 1A, B). 40 Hz response is decreased to about 24% below the control case, calculated as an average of 20 simulated control patients; 20 and 30 Hz responses are roughly the same as those of controls. This again was confirmed by re-running the model with 20 simulated schizophrenic patients.

To more formally test these effects, we ran a Group (control, schizophrenic)×Frequency (20 Hz, 30 Hz, 40 Hz) ANOVA. Both the main effects of Frequency (F [2, 80]=4812.6, p<0.001, Greenhouse-Geisser correction: ε=0.87) and Group (F [2, 80]=289.05, p<0.001, ε=0.87) were significant. Critically, these effects were qualified by a significant Group by Frequency interaction, driven by greatest group differences at 40 Hz (see FIG. 1D). Because groups differed in all three frequencies, a set of hierarchical regression analyses was run to test the specificity of the findings. Specifically, in the first regression, we entered power at 20 and 30 Hz in the first step, and Group (dummy-coded) in the second step, in order to predict power at 40 Hz. The model was significant, indicating that Group predicted 40 Hz activity when controlling for power at 20 and 30 Hz (ΔR2=0.101, ΔF [1], [38]=77.64, p<0.001). Critically, when entering 40 in the first step, Group predicted neither 30 Hz power (ΔR2=0.003, ΔF [1], [38]=0.81, p=0.375) nor 20 Hz power (ΔR2=0.025, ΔF [1], [38]=0.81, p=0.193). Thus, group differences were specific to 40 Hz.

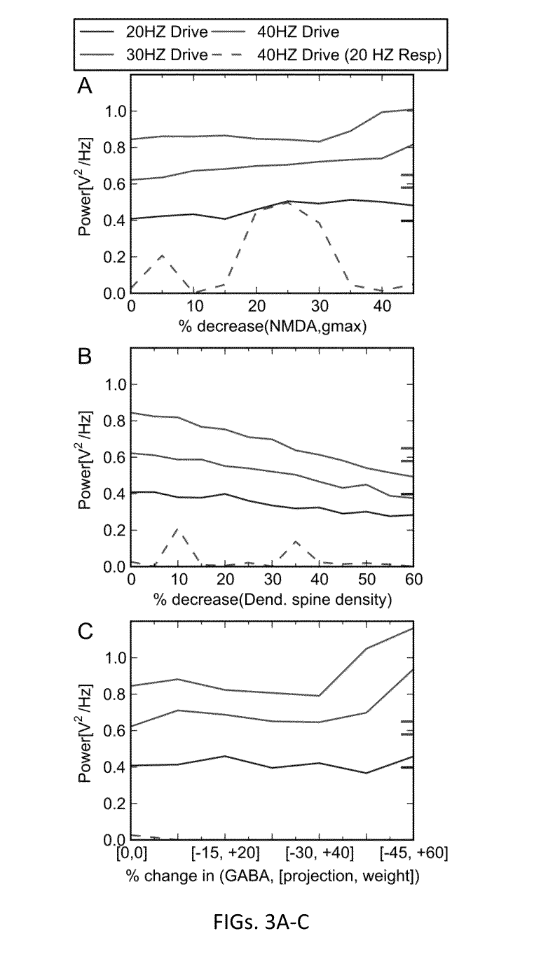

In an attempt to understand the relative contributions of each of these neural level abnormalities individually to the functioning of the system, we performed a “partial derivative” analysis for each. That is, we examined the overall behavior of the system in response to one lesion at a time, holding the others constant. The results are shown in FIGS. 3 and 4. Significantly, no single abnormality alone accounts for the findings.

### Example 3

### Analysis of Oscillatory Dynamics

What neural interactions caused the primary point, with a specific deficit in response to 40 Hz drive, to arise, and how did this differ from the secondary point? To answer this, we examined simulated EEG traces and histograms of spiking activity from both cases. Of note, for 40 Hz drive, the EEG traces of the primary point shows a depression of every other peak, effectively creating a mix of 20 and 40 Hz activity, and a decrease in the 40 Hz response (FIG. 5A-D). The spiking probability histograms for the primary and secondary points show averages over two cycles at a time, in an attempt to reveal differential contributions from inhibitory interneurons in alternating cycles. Notably, while both points produce a schizophrenic pattern of oscillatory activity when analyzed at the power spectrum level, there are clear differences in underlying neurophysiologic dynamics, as shown in FIGS. 5C and D. Panel C clearly shows alternating pyramidal cell activity across cycles; it also reveals a somewhat less marked alternation of PV+ cell activity, as well as modest cycle-to-cycle CR+ activity imbalance. Panel D (secondary point) shows a general damping down of pyramidal cell activity that is roughly constant across cycles, and little cycle-to-cycle variation in PV+ or CR+ activity.

### Example 4

### Simulation of Medication Effects

Negative Controls:

An important goal of this work is to develop a model that can identify novel pharmacologic agents that can potentially treat the symptoms of schizophrenia. Such a model should also be capable of rejecting current medications known to have no known antipsychotic efficacy. Therefore, when applied to the schizophrenic model they should not produce normalization of oscillatory powers. These then serve as “negative controls”. We chose the test agents described below based on the following considerations: (a) Their neurophysiologic effects are well-characterized, and they can therefore be included in the model in a rigorous manner. (b) There is a published literature documenting their non-effectiveness in the illness. (c) There is a history of clinical use, and their effects on (control) subject EEG activity are known.

For these trials, the primary point schizophrenic model, as defined above, is used as our test system. In separate trials, we apply the effects of phenyloin, an antiepileptic drug that has a specific effect at the Na+ channel (FIGS. 6A-B); nifedipine, an antihypertensive that acts by blocking calcium channels (FIGS. 7A-B); and ampakines, medications that allosterically bind to AMPA receptors and increase their activity [42]-[44], both by increasing maximum conductance and by increasing the decay time constant (FIG. 8). In no case does the agent correct the 40 Hz deficit. Moreover, when applied to our unaffected model, they produce EEG changes comparable to those seen in the clinical literature. This serves as additional confirmation of the validity of the computational model.

Virtual Medication Trials:

Many experimental medications for schizophrenia act through one particular mechanism of action. However, it is possible that adjustment of a number of cellular level “levers” would be necessary to return the system to a healthy equilibrium state. We examined five such effects, applying each to the model individually, and in combinations with others. Broadly, these mechanisms fall into two categories: those that can be effected with currently known medications (discussed under AMPA gmax, alpha2, and NMDA sections below); and those that, to the knowledge of these authors, cannot be implemented with any currently known agent (discussed under AMPA τ2 and CR+ projection below)—if effective, these would then represent potential targets for drug development efforts. The manner in which these were modeled is briefly described below, and summarized in Table 6.

TABLE 6
Parameter ranges used for simulated medications

| Parameter | Description | Units | Range of Values | Incr |
|---|---|---|---|---|
| AMPA gₘₐₓ | conductance of AMPA channel | % increase | 0, 20, 40, 60, 80 | 5 |
| alpha₂ | conductance of GABA channel, alpha₂ | % increase | 0, 15, 30, 45, 60 | 5 |
| | subtype | | | |
| NMDA | conductance of NMDA channel¹ | % increase | 0, 20, 40, 60, 80 | 5 |
| AMPA τ₂ | decay time constant of AMPA channel | msec | 1, 3, 5 | 3 |
| CR proj | weight of projection of CR cells on | % increase | 0, 20, 40, 60 | 4 |
| | postsynaptic targets | | | |

| | |
|---|---|
| total number of simulated trials: | 1,500 |

¹Resultant increase in intracellular Ca⁺⁺ also induces LTP; the quantitative manner in which this is implemented is described in the text, and is in addition to the effect shown here.
Using the schizophrenic model, simulated medication trials were run, systematically varying model parameters through the ranges shown. A total of 3 × 5 × 5 × 5 × 4 = 1,500 simulated trials were conducted.
Incr = number of increments.

AMPA Gmax.

The effect of drugs that boost AMPA current were modeled by increasing the maximum conductance (gmax) of the AMPA synaptic current. We did this in increments of 20%, increasing gmax from 0% to 80%.

Alpha2.

The experimental drug MK-0777 (also known as TPA-023) has partial agonist activity at GABA_{A} receptors, specifically acting at the α2 and α3 subtypes [45], and has shown partial effectiveness in treating some of the cognitive symptoms of schizophrenia [46]. These receptor subtypes are located on the initial segment of pyramidal cells, and are thought to be associated with the inhibitory projections of chandelier cells. While dissociation constants have been quantified [45], to our knowledge, MK-0777's quantitative effect on GABA channel conductance has not been. Electrophysiological studies with mutant mice (knock-in mice selectively expressing GABAA α2, α3, etc subtypes), has indicated that benzodiazepines can increase α2 and α3 conductance by as much as 50% [47]. Thus, to capture a plausible range of drug-induced conductance changes, we selectively increased the gmax of the GABA channels that synapse on the initial segment of pyramidal cells in increments of 15%, increasing gmax from 0 to 60%, in five gradations.

MK-0777 is one of the few cases in which a drug was tested in an experimental paradigm that involved schizophrenic patients and measurement of gamma band oscillations [46]. In this work, schizophrenic patients taking this drug showed a trend toward greater gamma band activity, which did not reach statistical significance at the p=0.05 level (their FIG. 1, p. 1589-90). To ensure that our model system behaved similarly, we implemented an MK-0777 effect alone, and observed a very modest increase in 40 Hz resonance within certain dose ranges, consistent with the experimental findings.

NMDA.

NMDA boosting drugs, such as D-serine, have potential benefit both because they increase NMDA current, and because resulting intracellular calcium increases enhance long term potentiation (LTP). To model the former, we increased gmax of the NMDA conductance in increments of 20%, to a maximum 80% increase, in five gradations. Single cell modeling that we carried out suggested that the ratio of percentage NMDA conductance increase:overall intracellular calcium concentration increase was approximately 2:1. While it is known that increases in intracellular Ca++ concentration trigger LTP, their precise quantitative relationship remains uncertain [48]-[50]. Detailed modeling work by Shouval et al [51] suggests a ratio of approximately 62%:29% (increase in calcium: degree of synaptic strengthening) (their FIG. 1, p. 10832). Based on this, for every NMDA channel, for each 20% increase in NMDA channel conductance, we also increased the synaptic weight factor by 4.7%.

AMPA τ2.

Modeling work described above suggests that significantly increasing the AMPA conductance decay time (τ2), in the manner of certain ampakines, does not improve model performance. However, in exploratory modeling work, we found that decreasing this parameter seemed to have positive effects. Therefore, we used τ2 values of 1, 3 (control), and 5 msec.

CR+ Projections.

Exploratory runs of the schizophrenic model indicated that the calretinin cell projections (which impinge only on other interneurons) have a general quantitative modulatory role: increasing the weight of these projections tended to produce greater network activity overall (including 40 Hz oscillatory behavior) and decreasing them lead to generalized decreases in activity. Based on this, we adjusted upward the synaptic weight factor of the CR+ cells onto their postsynaptic targets, increasing it from 0 to 60%, in four gradations.

We ran each of the above effects alone, and in combination with all other effects, for a total of 5×5×5×3×4=1,500 trials (Table 6). For each trial, the model was driven at 20, 30, and 40 Hz, as described in our previous trials investigating schizophrenic pathology. To the extent that a simulated medication specifically increased 40 Hz power response to 40 Hz drive, it was considered effective. That is, if a treated schizophrenic model exactly replicated control model behavior, it would receive a score of 1.0; the score was decreased to the extent that it departed from this. Trials that did not produce 20 Hz and 30 Hz power within 10% of control were marked as failed trials, and received a score of 0. Thus, a simulated drug that boosted all frequencies indiscriminately would not be considered effective.

In total, 97 virtual medications, or 6.5% of the 1,500 tried, produced non-zero scores. 24 received scores 0.90 or higher. The characteristics of these drugs are shown via histograms in FIG. 9.

It can be seen that of these top performing medications, many decreased AMPA τ2 and modestly increased NMDA activity. Clearly, a number of these effects may have interacted to produce desirable outcomes. To understand this at a fine grained level, we ran a three-way analysis of variance on the 97 scored virtual medications; results are shown in Table 7, with effects showing significance at a level of p<0.001 indicated. Of note, (decreasing) AMPA τ2 emerged as a significant effect, alone and in combination. A very significant interaction between AMPA τ2 and CR+ projection strength also emerged.

TABLE 7
Analysis of variance of model response to drug effects

| Simulated drug effect | F value | p value |
|---|---|---|
| alpha₂ | 0.01 | 0.916 |
| AMPA gₘₐₓ | 1.76 | 0.185 |
| AMPA τ₂ | 25.14 | 6.00E−07 * |
| NMDA | 21.17 | 4.55E−06 * |
| CR proj | 0.35 | 0.554 |
| alpha₂:AMPA gₘₐₓ | 0.92 | 0.338 |
| alpha₂:AMPA τ₂ | 1.23 | 0.268 |
| AMPA gₘₐₓ:AMPA τ₂ | 13.43 | 2.56E−04 * |
| alpha₂:NMDA | 6.52 | 0.011 |
| AMPA gₘₐₓ:NMDA | 5.24 | 0.022 |
| AMPA τ₂:NMDA | 0.01 | 0.914 |
| alpha₂:CR proj | 1.78 | 0.182 |
| AMPA gₘₐₓ:CR proj | 10.44 | 0.001 |
| AMPA τ₂:CR proj | 68.18 | 3.29E−16 * |
| NMDA:CR proj | 1.91 | 0.167 |
| alpha₂:AMPA gₘₐₓ:AMPA τ₂ | 0.84 | 0.358 |
| alpha₂:AMPA gₘₐₓ:NMDA | 0.48 | 0.490 |
| alpha₂:AMPA τ₂:NMDA | 1.53 | 0.217 |
| AMPA gₘₐₓ:AMPA τ₂:NMDA | 1.57 | 0.211 |
| alpha₂:AMPA gₘₐₓ:CR proj | 0.37 | 0.545 |
| alpha₂:AMPA τ₂:CR proj | 4.19 | 0.041 |
| AMPA gₘₐₓ:AMPA τ₂:CR proj | 1.11 | 0.292 |
| alpha₂:NMDA:CR proj | 2.69 | 0.101 |
| AMPA gₘₐₓ:NMDA:CR proj | 4.21 | 0.040 |
| AMPA τ₂:NMDA:CR proj | 13.34 | 2.70E−04* |

* p < 0.001
Using the wellness metric (see text), as the outcome variable, 97 of 1,500 simulated medications produced non-zero values. ANOVA of this output, using parameters for drug effects as factors, is shown. Highly significant effects (p < 0.001) are indicated.
CR proj = CR+ projection.

### REFERENCES

1. Stilo S A, Murray R M (2010) The epidemiology of schizophrenia: replacing dogma with knowlege. Dialogues in Clinical Neuroscience 12: 305-315
2. Henderson D C, Cagliero E, Copeland P M, Borba C P, Evins E, et al. (2005) Glucose metabolism in patients with schizophrenia treated with atypical antipsychotic agents: a frequently sampled intravenous glucose tolerance test and minimal model analysis. Archives of General Psychiatry 62: 19-28
3. Coyle J T, Balu D, Benneyworth M, Basu A, Roseman A (2010) Beyond the dopamine receptor: novel therapeutic targets for treating schizophrenia Dialogues in Clinical Neuroscience 12: 359382
4. Heckers S, Konradi C (2010) Hippocampal pathology in schizophrenia. Current Topics in Behavioral Neurosciences 4: 529-553
5. Benes F, Berretta S (2001) GABAergic interneurons: implications for understanding schizophrenia and bipolar disorder. Neuropsychopharmacology 25: 1-27
6. Coyle J (2006) Glutamate and schizophrenia: beyond the dopamine hypothesis Cellular and Molecular Neurobiology 4-6: 365-384
7. Greene R (2001) Circuit analysis of NMDAR hypofunction in the hippocampus, in vitro, and psychosis of schizophrenia. Hippocampus 11: 569-577
8. Hoffman R E, McGlashan T H (2003) Schizophrenia: From psychodynamic to neurodynamic theories. In: Sadock J S, Sadock V A, editors. Kaplan and Sadock's Synopsis of Psychiatry. 9th ed. Philadelphia, Pa.: Lippincott Williams and Wilkins
9. Feinberg I (1982) Schizophrenia: caused by a fault in programmed synaptic elimination during adolescence? Journal of Psychiatric Research 17: 319-334
10. Beggs J (2008) The criticality hypothesis: how local cortical networks might optimize information processing. Philosophical Transactions of the RoyalSociety A: Physical, Mathematical and Engineering Sciences 366: 329-343
11. Lisman J (2012) Excitation, inhibition, local oscillations, or large-scale loops: what causes the symptoms of schizophrenia? Current Opinion in Neurobiology 22: 537-544
12. Cho R Y, Konecky R O, Carter C S (2006) Impairments in frontal cortical gamma synchrony and cognitive control in schizophrenia. Proceedings of the National Academy of Sciences of the USA 103: 19878-19883
13. Ferrarelli F, Massimini M, Peterson M J, Riedner B A, Lazar M, et al. (2008) Reduced evoked gamma oscillations in the frontal cortex in schizophrenia patients: a TMS/EEG study. American Journal of Psychiatry 165: 996-1005
14. Hirano S, Hirano Y, Maekawa T, Obayashi C, Oribe N, et al. (2008) Abnormalneural oscillatory activity to speech sounds in schizophrenia: a magnetoencephalography study. Journal of Neuroscience 28: 4897-4903
15. Spencer K M, Salisbury D F, Shenton M E, McCarley R W (2008) Gamma-band auditory steady-state responses are impaired in first episode psychosis. Biological Psychiatry 64: 369-375
16. Teale P, Collins D, Maharajh K, Rojas D C, Kronberg E, et al. (2008) Cortical source estimates of gamma band amplitude and phase are different in schizophrenia. Neuroimage 42: 1481-1489
17. Light G A, Hsu J L, Hsieh M H, Meyer-Gomes K, Sprock J, et al. (2006) Gamma band oscillations reveal neural network cortical coherence dysfunction in schizophrenia patients. Biological Psychiatry 60: 1231-1240
18. Kopell N, Ermentrout G, Whittington M, Traub R (2000) Gamma rhythms and beta rhythms have different synchronization properties. Proceedings of the National Academy of Sciences of the USA 97: 1867-1872
19. Wang X (2006) Toward a prefrontal microcircuit model for cognitive deficits in schizophrenia. Pharmacopsychiatry 39 Suppl 1: S80-87
20. Durstewitz D, Seamans J (2008) The dual-state theory of prefrontal cortex dopamine function with relevance to catechol-o-methyltransferase genotypes and schizophrenia. Biological Psychiatry 64: 739-749
21. Hoffman R E (1987) Computer simulations of neural information processing and the schizophrenia-mania dichotomy. Archives of General Psychiatry 44: 178-188
22. Rolls E T, Loh M, Deco G, Winterer G (2008) Computational models of schizophrenia and dopamine modulation in the prefrontal cortex. Nature Reviews Neuroscience 9: 696-709
23. Siekmeier P J, Hasselmo M E, Howard M W, Coyle J (2007) Modeling of context dependent retrieval in hippocampal region CA1: Implications for cognitive function in schizophrenia. Schizophrenia Research 89: 177-190
24. Traub R D, Jefferys J G, Miles R, Whittington M A, Toth K (1994) A branching dendritic model of a rodent CA3 pyramidal neurone. Journal of Physiology(London) 481: 79-95
25. Traub R D, Miles R (1995) Pyramidal cell-to-inhibitory cell spike transduction explicable by active dendritic conductances in inhibitory cell. Journal of Computational Neuroscience 2: 291-298
26. Siekmeier P J (2009) Evidence of multistability in a realistic computer simulation of hippocampus subfield CA1. Behavioural Brain Research 200: 220-231
27. Tsai G, Passani L, Slusher B, Carter R, Baer L, et al. (1995) Abnormal excitatory neurotransmitter metabolism in schizophrenic brains. Archives of General Psychiatry 52: 829-836
28. Bergeron T, Coyle J, Tsai G, Greene R (2005) NAAG reduces NMDA receptor current in CA1 hippocampal pyramidal neurons of acute slices and dissociated neurons. Neuropsychopharmacology 30: 7-16
29. Law A, Deakin J (2001) Asymmetrical reductions of hippocampal NMDAR1 glutamate receptor mRNA in the psychoses. Neurochemistry 12: 2971-2974
30. Harrison P, Law A, Eastwood S (2003) Glutamate receptors and transporters in the hippocampus in schizophrenia Annals of the New York Academy of Sciences 1003: 94-101
31. Hoffman R E, Dobscha S K (1989) Cortical pruning and the development of schizophrenia: a computer model. Schizophrenia Bulletin 15: 477-490
32. Law A, Weickert C, Hyde T, Kleinman J, Harrison P (2004) Reduced spinophilin but not microtubule-associated protein 2 express in the hippocampal formation in schizophrenia and mood disorders: Molecular evidence for a pathology of dendritic spines. American Journal of Psychiatry 161: 1848-1855
33. Garey L J, Ong W Y, Patel T S, Kanani M, Davis A, et al. (1998) Reduced dendritic spine density on cerebral cortical pyramidal neurons in schizophrenia Journal of Neurology, Neurosurgery, and Psychiatry 65: 446-453
34. Devito L, Balu D, Kanter B, Lykken C, Basu A, et al. (2011) Serine racemase deletion disrupts memory for order and alters cortical dendritic morphology Genes Brain Behav 10: 210-222
35. Heckers S, Stone D, Walsh J, Shick J, Koul P, et al. (2002) Differential Hippocampal Expression of Glutamic Acid Decarboxylase 65 and 67 Messenger RNA in Bipolar Disorder and Schizophrenia. Archives of General Psychiatry 59:521-529
36. Bird E, Barnes J, L L I, Spokes E, Mackay A, et al. (1977) Increased brain dopamine and reduced glutamic acid decarboxylase and choline acetyltransferase activity in schizophrenia and related psychoses. The Lancet 2:1157-1158
37. Fatemi S, Earle J, McMenomy T (2000) Reduction in reelin immunoreactivity in hippocampus of subjects with schizophrenia, bipolar disorder and major depression. Molecular Psychiatry 5: 654-663
38. Torrey E, Barci B, Webster M, Bartko J, Meador-Woodruff J, et al. (2005) Neurochemical markers for schizophrenia, bipolar disorder, and major depression in postmortem brains. Biological Psychitry 57: 252-260
39. Benes F M, Khan Y, Vincent S L, Wickramasinghe R (1996) Differences in the subregional and cellular distribution of GABAA receptor binding in the hippocampal formation of schizophrenic brain. Synapse 22: 338-349
40. Lisman J, Coyle J, Green R, Javitt D, Benes F, et al. (2008) Circuit-based framework for understanding neurotransmitter and risk gene interactions in schizophrenia. Trends in Neuroscience 31: 234-242
41. Kwon J S, O'Donnell B F, Wallenstein G V, Greene R W, Hirayasu Y, et al (1999) Gamma frequency-range abnormalities to auditory stimulation ins chizophrenia. Archives of General Psychiatry 56: 1001-1005
42. Johansen T H, Chaudhary A, Verdoorn T A (1995) Interactions among GYKI-52466, cyclothiazide, and aniracetam at recombinant AMPA and kainite receptors. Mol Pharmacol 48: 946-955
43. Arai A C, Kessler M, Rogers G, Lynch G (2000) Effects of the potent ampakine CX614 on hippocampal and recombinant AMPA receptors: interactions with cyclothiazide and GYKI 52466. Mol Pharmacol 58: 802-813
44. Arai A C, Xia Y F, Rogers G, Lynch G, Kessler M (2002) Benzamide-type AMPA receptor modulators form two subfamilies with distinct modes of action. J Pharmacol Exp Ther 303: 1075-1085
45. Atack J R, Wong D F, Fryer T D, Ryan C, Sanabria S, et al. (2010) Benzodiazepine binding site occupancy by the novel GABAA receptor subtype-selective drug 7-(1,1-Dimethylethyl)-6-(2-ethyl-2H-1,2,4-triazol-3-ylmethoxy)-3-(2-fluorophenyl)-1,2,4-triazolo[4,4-b]pyridazine (TPA023) in rats, primates, and humans. The Journal of Pharmacology and Experimental Therapeutics 332: 17-25
46. Lewis D A, Cho R Y, Carter C S, Eklund K, Forster S, et al. (2008) Subunit selective modulation of GABA type A receptor neurotransmission and cognition in schizophrenia. American Journal of Psychiatry 165: 1585-1593
47. Prenosil G A, Gasser E M, Rudolph U, Keist R, Fritschy J-M, et al. (2006) Specific subtypes of GABAA receptors mediate phasic and tonic forms of inhibition in hippocampal pyramidal neurons. Journal of Neurophysiology 96:846-857
48. Kotaleski J H, Blackwell K T (2010) Modeling the molecular mechanisms of synaptic plasticity using systems biology approaches. Nature Reviews Neuroscience 11: 239-251
49. Lynch M A (2004) Long-term potentiation and memory. Physiology Reviews 84:87-136
50. Cavazzini M, Bliss T, Emptage N (2005) Ca2+ and synaptic plasticity. Cell Calcium 38: 355-367
51. Shouval H Z, Bear M F, Cooper L N (2002) A unified model of NMDA receptor dependent bidirectional synaptic plasticity. Proc Natl Acad Sci USA 99:10831-10836
52. Uhlhaas P J, Singer W (2010) Abnormal neural oscillations and synchrony in schizophrenia. Nat Rev Neurosci 11: 100-113
53. Meyer-Lindenberg A, Weinberger D (2006) Intermediate phenotypes and genetic mechanisms of psychiatric disorders. Nature Reviews Neuroscience 7:818-827
54. Vierling-Claassen D, Siekmeier P, Stufflebeam S, Kopell N (2008) Modeling GABA alterations in schizophrenia: a link between impaired inhibition and altered gamma and beta range auditory entrainment. Journal of Neurophysiology 99: 2656-2671
55. Kissler J, Muller M M, Fehr T, Rockstroh B, Elbert T (2000) MEG gamma band activity in schizophrenia patients and healthy subjects in a mental arithmetic task and at rest. Clinical Neurophysiology 111: 2079-2087
56. Beasley C L, Zhang Z J, Patten I, Reynolds G P (2002) Selective deficits in prefrontal cortical GABAergic neurons in schizophrenia defined by the presence of calcium-binding proteins. Biological Psychiatry 52: 708-715
57. Hashimoto T, Bazmi H H, Mimics K, Wu Q, Sampson A R, et al. (2008) Conserved regional patterns of GABA-related transcript expression in the neocortex of subjects with schizophrenia. American Journal of Psychiatry 165:497-489
58. Knable M B, Barci B M, Webster M J, Meador-Woodruff J, Torrey E F (2004) Molecular abnormalities of the hippocampus in severe psychiatric illness: postmortem findings from the Stanley Neuropathology Consortium. Molecular Psychiatry 9: 609-620
59. Berk R, Bickel P, Campbell K, Fovell R, Keller-McNulty S, et al. (2007) Workshop on statistical approaches for the evaluation of complex computer models. Statistical Science 17: 173-192
60. Bayarri M, Berger J, Cafeo G, Garcia-Donato F, Liu F, et al. (2007) Computer model validation with functional output. The Annals of Statistics 35: 1874-1906
61. Easterling R, Berger J. Statistical foundations for the validation of computer models. In: Pace D, Stevenson S, editors; 2002. Society for Modeling and Simulation International
62. Kilpatrick C J, Wanwimolruk S, Wing L M (1984) Plasma concentrations of unbound phenyloin in the management of epilepsy. British Journal of Clinical Pharmacology 17: 539-546
63. Lampl I, Schwindt P, Crill W (1998) Reduction of Cortical Pyramidal Neuron Excitability by the Action of Phenyloin on Persistent Na+ Current. Journal of Pharmacology and Experimental Therapeutics 284: 228-237
64. Fink M, Irwin P, Sannita W, Papakostas Y, Green M A (1979) Phenyloin: EEG effects and plasma levels in volunteers. Therapeutic Drug Monitoring 1: 93-103
65. Chao T I, Alzheimer C (1995) Effects of phenyloin on the persistent Na+ current of mammalian CNS neurons. NeuroReport 6: 1778-1780
66. Herkes G K, Lagerlund T D, Sharbrough F W, Eadie M J (1993) Effects of antiepileptic drug treatment on the background frequency of EEGs in epileptic patients. Journal of Clinical Neurophysiology 10: 210-216
67. Van Wieringen A, Binnie C D, Meijer J W A, Peck A W, de Vries J (1989) Comparison of the effects of lamotragine and phenyloin on the EEG power spectrum and cortical brainstem-evoked responses of normal human volunteers Neuropsychobiology 21: 157-169
68. Cunningham M O, Whittington M A, Bibbig A, ZRoopun A, LeBean R E N, et al (2004) A role for fast rhythmic bursting neurons in cortical gamma oscillations in vitro. Proceedings of the National Academy of Sciences of the United States of America 101: 7152-7157
69. Deutz N E P, Chamuleau R A, Bovee W M, VanderWerf A J (1986) Effects of nimodipine on EEG and 31P-NMR spectra during and after in complete forebrian ischemia in the rat. European Journal of Pharmacology 125: 429-435
70. Denolle T, Sassano P, Allain H, Bentue-Ferrer D, Breton S, et al. (2002) Effects of nicardipine and clonidine on cognitive functions and electroencephalography in hypertensive patients. Fundamental and Clinical Pharmacology 16: 527-535
71. Sannita W G, Garbarino S, Gesino D, Massimilla S, Ogliastro C (1999) Plasma concentrations and CNS effects of Ca antagonists darodpine and nimodipine after single-dose oral administration to healthy volunteers. Neuropsychobiolgy 40: 158-170
72. Johansen T H, Chaudhary A, Verdoorn T A (1995) Interactions among GYKI-52488, cyclothiazide, and anifacetam at recombinant AMPA and kainite receptors. Molecular Pharmacology 48: 946-955
73. Arai A C, Kessler M, Rogers G, Lynch G (2000) Effects of the potent amapkine CX614 on hippocampal and recombinant AMPA receptors: interactions with cyclothiazide and GYKI 52466. Molecular Pharmacology 58: 802-813
74. Arai A C, Xia Y-F, Gogers G, Lynch G, Kessler M (2002) Benzamide-type AMPA receptor modulators form two subfamilies with distinct modes of action The Journal of Pharmacology and Experimental Therapeutics 303: 1075-1085
75. Grove S J A, Jamieson C, Maclean J K F, Morrow J A, Rankovic Z (2010) Positive allosteric modulators of the alpha-amino-3-hydroxy-5-methyl-4-isoxazolepropionicacid (AMPA) receptor. Journal of Medicinal Chemistry 53: 7271-7279
76. Goff D C, Lamberti J S, Leaon A C, Green M F, Miller A L, et al. (2008) A placebo-controlled add-on trial of amapkine, CX516, for cognitive deficits in schizophrenia. Neuropsychopharmacology 33: 465-472

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a candidate agent for the treatment of schizophrenia, the method comprising:
providing a sample comprising a cell expressing functional 2-amino-3-(3-hydroxy-5-methyl-isoxazol-4-yl)propanoic acid (AMPA) channels;
contacting the sample with a test compound;
measuring the decay time constant (tau2) of the AMPA conductance in response to stimulation in the presence, and optionally in the absence, of the test compound; and
selecting as a candidate agent a test compound that decreases the tau2 of the AMPA conductance.

2. The method of claim 1, comprising selected as a candidate agent a test compound that decreases the tau2 of the AMPA conductance to 3 msec or less.

3. The method of claim 1, wherein the decay time constant is measured electrophysiologically or by imaging of a calcium imaging agent.

4. A method of identifying a candidate agent for the treatment of schizophrenia, the method comprising:
providing a sample comprising a neural network comprising a calretinin-positive (CR+) GABAergic interneuron, and at least one postsynaptic neuron or interneuron receiving synaptic input from the CR+ interneuron;
contacting the sample with a test compound;
stimulating the CR+ interneuron and measuring the response in the postsynaptic neuron or interneuron in the presence and absence of the test compound; and
selecting as a candidate agent a test compound that increases the response in the postsynaptic neuron or interneuron.

5. The method of claim 4, wherein the neural network is a neocortical, allocortical, or hippocampal brain slice, or an in vitro neural network.

6. The method of claim 5, wherein the brain slice is from an animal model of schizophrenia, or from a normal non-schizophrenic animal.

7. The method of claim 5, wherein the in vitro neural network comprises primary neurons from an animal model of schizophrenia, or from a normal non-schizophrenic animal.

8. The method of claim 4, wherein measuring the response in the postsynaptic neuron or interneuron comprises measuring one or more of: long term potentiation at the postsynaptic synapse; short term potentiation; conductance change; response to paired pulses; inhibitory postsynaptic current (IPSC); and inhibitory postsynaptic potential (IPSP) in the postsynaptic neuron or interneuron.

9. A method of identifying a candidate agent for the treatment of schizophrenia, the method comprising:
providing a sample comprising a postsynaptic neuron or interneuron that receives synaptic input from a calretinin-positive (CR+) GABAergic interneuron;
identifying a combination of GABAA receptor subunits expressed in the postsynaptic neuron or interneuron; and
selecting a drug that is a specific agonist of GABAA receptors comprising the subunits expressed in the postsynaptic neuron or interneuron as a candidate agent for the treatment of schizophrenia.

10. The method of claim 9, wherein selecting a drug that is a specific agonist of GABAA receptors comprising the subunits expressed in the postsynaptic neuron as a candidate agent for the treatment of schizophrenia comprises:
expressing the subunits expressed in the postsynaptic neuron or interneuron in a mammalian cell to form functional GABAA receptors;
contacting the mammalian cell with a test compound;
detecting conductance through a GABAA receptor in the cell in the presence of the test compound;
selecting as a candidate compound a test compound that increases conductance as compared to conductance in the absence of the test compound.

11. The method of claim 1, further comprising:
administering the selected candidate compound to an animal model of schizophrenia;
monitoring one or more symptoms of schizophrenia in the animal model; and
selecting as a candidate therapeutic agent a candidate compound that improves one or more symptoms of schizophrenia in the animal model.

12. The method of claim 4, further comprising:
administering the selected candidate compound to an animal model of schizophrenia;
monitoring one or more symptoms of schizophrenia in the animal model; and
selecting as a candidate therapeutic agent a candidate compound that improves one or more symptoms of schizophrenia in the animal model.

13. The method of claim 9, further comprising:
administering the selected candidate compound to an animal model of schizophrenia;
monitoring one or more symptoms of schizophrenia in the animal model; and
selecting as a candidate therapeutic agent a candidate compound that improves one or more symptoms of schizophrenia in the animal model.

14. A method of treating schizophrenia in a subject, the method comprising administering a therapeutically effective amount of a combination of compounds comprising:
(a) an NMDA agonist and a GABAA-alpha 2 agonist; or
(b) an NMDA agonist and an AMPAkine.

15. The method of claim 14, wherein the NMDA agonist is selected from the group consisting of UBP646, UBP512, UBP551, CIQ, Glycine, D-cycloserine, glycine type I (GlyT1) transporter inhibitors, and D-serine.

16. The method of claim 15, wherein the glycine type I (GlyT1) transporter inhibitor is sarcosine (N-methylglycine) or RG1678.

17. The method of claim 14, wherein the GABAA-alpha 2 agonist is MK-0777, TPA023B or MRK-409.

18. The method of claim 14, wherein the AMPAkine is piracetam, aniracetam, CX516, CX717, CX691 (faramptor), LY451395 or CX546.
